(19) United States
(12) Patent Application Publication
(10) Pub. No.: US 2007/0190070 A1
(43) Pub. Date: Aug. 16, 2007

(54) Methods of using and compositions comprising selective cytokine inhibitory drugs for the treatment and management of disorders of the central nervous system

(76) Inventors: Jerome B. Zeldis, Princeton, NJ (US); Peter H. Schafer, Somerset, NJ (US)

Correspondence Address: JONES DAY, 222 EAST 41ST ST, NEW YORK, NY, 10017, US

(21) Appl. No.: 10/590,575

(22) PCT Filed: Sep. 3, 2004
(86) PCT No.: PCT/US04/28742

§ 371 Date: Aug. 22, 2006

(51) Int. Cl. A61K 31/4704; A61K 31/425; A61K 31/4035

(52) U.S. Cl. 424/184.1; 514/417; 514/662; 514/309; 514/373

(57) ABSTRACT

Methods of treating, preventing and/or managing central nervous system disorders, such as Parkinson disease, Alzheimer disease, mild cognitive impairment, Huntington disease, Amytophic Lateral Sclerosis, depression and defective long-term memory, and related syndromes are disclosed. Specific methods encompass the administration of a selective cytokine inhibitory drug, or a pharmaceutically acceptable salt, solvate, hydrate, stereoisomer, clathrate, or prodrug thereof, alone or in combination with a second active ingredient. Pharmaceutical compositions, single unit dosage forms, and kits suitable for use in methods of the invention are also disclosed.

### 1. FIELD OF THE INVENTION

This invention relates, in part, to methods of treating, preventing and/or managing central nervous system disorders, including but not limited to, Parkinson disease, Alzheimer disease, mild cognitive impairment, Huntington disease, Amyotrophic Lateral Sclerosis, depression and defective long-term memory, and related disorders which comprise the administration of a selective cytokine inhibitory drug, or a pharmaceutically acceptable salt, solvate, hydrate, stereoisomer, clathrate, or prodrug thereof.

### 2. BACKGROUND OF THE INVENTION

Central nervous system disorders affect a wide range of the population with differing severity. Generally, one major feature of this class of disorders includes the significant impairment of cognition or memory that represents a marked deterioration from a previous level of functioning. Dementia, for example, is characterized by several cognitive impairments including significant memory deficit and can stand alone or be an underlying characteristic feature of a variety of diseases, including Alzheimer disease, Parkinson disease, Huntington disease, and Multiple Sclerosis to name but a few. Other central nervous system disorders include delerium, or disturbances in consciousness that occur over a short period of time, and amnestic disorder, or discreet memory impairments that occur in the absence of other central nervous system impairments.

2.1 Parkinson Disease

Parkinson disease (PD) is the second most common neurodegenerative disease and affects approximately 1% of the population over 50 years of age. Polymeropoulos et al., 1996, Science 274: 1197-1198. Approximately one million Americans suffer from PD, and each year 50,000 individuals are diagnosed with the disorder. Olson, L., 2000, Science 290:721-724. Because early symptoms of PD may go unrecognized, perhaps as many as 5 to 10% of individuals over 60 years of age may have the illness. Olson, L., 2000, Science 290:721-724.

It has been known since the 1960s that loss of dopamine neurons in the nigrostriatal pathway of the brain results in the motor abnormalities characteristic of PD. Typical onset of PD occurs in mid to late adulthood with progressive clinical features. Some of the physical manifestations of PD include resting tremors, muscular rigidity, postural instability, and dementia. Pathologic characteristics of PD include a loss of dopaminergic neurons in the substantia nigra (SN) as well as the presence of intracellular inclusions or Lewy Bodies in surviving neurons in various areas of the brain. Nussbaum, R. L. and Polymeropoulos, M. H., 1997, Hum. Molec. Genet. 6: 1687-1691. Interestingly, many other diseases have parkisonian motor features. The motor symptoms in PD are generally thought to result from the deficiency or dysfunction of dopamine or dopaminergic neurons in the substantia nigra. Nussbaum, R. L., Polymeropoulos, M. H., 1997, Hum. Molec. Genet. 6: 1687-1691. Evidence has also suggested that molecular chaperones, specifically heat shock proteins, HSP70 and HSP40, may play a role in PD progression. Auluck et al., 2002, Science 295: 865-868.

Much controversy exists regarding the etiology of PD, and there is evidence that both genetic and environmental factors may contribute to the disease. A study of the nuclear families of 948 PD cases concluded that a rare major mendelian inheritance gene, that influences age of onset, exists. Maher et al., 2002, Am. J. Med. Genet. 109: 191-197. This study also suggested the existence of a gene that influences susceptibility. Other evidence also suggests that environmental factors may be more significant than genetic factors in contributing to PD. Calne et al., 1987, Canad. J. Neurol. Sci. 14: 303-305. Researchers have concluded that most cases of PD are caused by environmental factors superimposed on a background of slow and sustained neuronal loss due to aging. Calne, D. B. and Langston, J. W., 1993, Lancet II 1457-1459. While the etiology remains unclear, it is likely that both genetic and environmental factors contribute to PD, and that environmental factors act upon genetic susceptibility to cause the disease. Recent evidence in animal models of Parkinson disease, suggests that anti-inflammatory agents inhibit dopaminergic cell death. McGeer et al., 2001, B. C. Med. J. 43:138-141.

While a cure is not currently available for Parkinson disease, traditional treatment has focused on responding to the effect of dopamine loss in the brain. Therapy using dopamine precursor, levodopa, became the treatment of choice when it was discovered that the compound could alleviate PD symptoms, thereby improving the quality of life for affected individuals. Unfortunately, it has become clear that long-term levodopa administration can have side affects. Caraceni et al., 1994 Neurology, 41:380. A variety of therapeutic strategies have been developed for the treatment of PD. MPTP, a neurotoxin known to specifically damage dopamine neurons, is commonly used as a model for the effects of PD. In one study, investigators used lentiviral vectors to deliver glial cell line derived neurotrophic factor (GDNF) to the striatum and SN of rhesus monkeys that had been treated one week prior with MPTP. Kordower et al., 2000, Science 290: 767-773. GDNF is known to have trophic effects upon degenerating nigrostriatal neurons in nonhuman primate models of Parkinson disease. Results of the study showed that GDNF augmented dopaminergic function in aged monkeys and reversed functional deficits and prevented nigrostriatal degeneration in monkeys that had been treated with MPTP. It was also noted that GDNF treatment reversed motor deficits in MPTP treated monkeys. This study also concluded that GDNF delivery could prevent nigrostriatal degeneration and induce regeneration of neurons in primate models of PD. Kordower et al., 2000, Science 290: 767-773.

Another study, using electrical inhibition and pharmacologic silencing of the subthalmic nucleus (STN), demonstrated that the alteration of basal ganglia network activity could improve motor network activity in PD, presumably by suppressing the firing activity of neurons in the SN. Luo et al., 2002, Science 298: 425-429. Investigators used an adeno-associated virus to transduce excitatory glutaminergic neurons in the rat STN with glutamic acid decarboxylase (GAD) to demonstrate that the change provided neuroprotection to the dopaminergic cells from toxic insults. Interestingly, rats with the transduced gene also showed significant improvement from parkinsonian phenotypes.

The selective PDE4 inhibitors Ro-20 1724 and SDZ-MNS 949, in the presence of the adenylate cyclase activator forskolin, have been shown to stimulate uptake of dopamine by rat mesencephalonic neurons in vitro (Hulley et al., J Neural Transm Suppl, 46:217-228, 1995). In these studies, elevation of cAMP by the addition of dibutyryl cAMP or forskolin protected dopaminergic neurons from the neurotoxic effects of MPP′ (1-methyl-4-phenyl pyridinium ion). These PDE4 inhibitors were shown to reduce dopamine depletion in the striatum and reduce loss of tyrosine hydroxylase-immunopositive neurons in the substantia nigra of C57BL/6 mice injected with MPTP (Hulley et al., Eur J Neurosci, 7:2431-2440, 1995). Therefore, PDE4 inhibitors have shown efficacy in the MPTP mouse model of PD, and based on in vitro studies, the mechanism of action is believed to at least partially involve a direct neuroprotective effect.

Recently, two groups have studied the role of TNF-α, receptors in the MPTP mouse model of PD. In one study, mice deficient in both forms of the TNF-α receptor (TNFR1 and TNFR2) were found to have decreased striatal dopamine levels and increased dopamine turnover (Rousselet et al., Exp Neurol, 177:183-192, 2002). In a separate study, TNFR1 and TNFR2 double knockout mice were completely protected against dopaminergic neurotoxicity of MPTP (Sriram et al., Faseh J 16:1474-1476, 2002). Therefore, it appears that TNF-α mediates neurotoxicity in this animal model of PD.

Further, J. D. Parkes et al. have investigated the anti-parkinsonian action of PDE4 inhibitor Rolipram in patients with PD. J. D. Parkes et al., 1984, Advances in Neurology, Vol. 40, 563-564. The effects of Rolipram were also assessed in a double-blind trial versus placebo in patients with PD already under treatment. Casacchia et al., Pharmacological Research Communications, Vol. 15, No. 3, 1983, 329-330. Contrary to other findings with specific phosphodiesterase inhibitors, no significant deterioration of the therapeutic action of dopamine against Lisuride was noted with Rolipram at the dose of 3 mg per day. Id. The dose-limiting side effect of nausea encountered with the PDE4 inhibitor Rolipram in Phase II trials of PD has significantly reduced its potential use.

2.2 Alzheimer Disease

Alzheimer disease (AD) is an increasingly prevalent form of neurodegeneration that accounts for approximately 50%-60% of the overall cases of dementia among people over 65 years of age. It currently affects an estimated 15 million people worldwide and owing to the relative increase of elderly people in the population its prevalence is likely to increase over the next 2 to 3 decades. Alzheimer disease is a progressive disorder with a mean duration of around 8.5 years between onset of clinical symptoms and death. Death of pyramidal neurons and loss of neuronal synapses in brains regions associated with higher mental functions results in the typical symptoms, characterized by gross and progressive impairment of cognitive function (Francis et al., 1999, J. Neurol. Neurosurg. Psychiatry 66:137-47). Alzheimer disease is the most common form of both senile and presenile dementia in the world and is recognized clinically as relentlessly progressive dementia that presents with increasing loss of memory, intellectual function and disturbances in speech (Merritt, 1979, A Textbook of Neurology, 6^{th} edition, pp. 484-489 Lea & Febiger, Philadelphia). The disease itself usually has a slow and insidious progress that affects both sexes equally, worldwide. It begins with mildly inappropriate behavior, uncritical statements, irritability, a tendency towards grandiosity, euphoria and deteriorating performance at work; it progresses through deterioration in operational judgment, loss of insight, depression and loss of recent memory; it ends in severe disorientation and confusion, apraxia of gait, generalized rigidity and incontinence (Gilroy & Meyer, 1979, Medical Neurology, pp. 175-179 MacMillan Publishing Co.).

The etiology of Alzheimer disease is unknown. Evidence for a genetic contribution comes from several important observations such as the familial incidence, pedigree analysis, monozygotic and dizygotic twin studies and the association of the disease with Down's syndrome (for review see Baraitser, 1990, The Genetics of Neurological Disorders, 2^{nd} edition, pp. 85-88). Nevertheless, this evidence is far from definitive and it is clear that one or more other factors are also required. Elevated concentrations of aluminum have been found in the brains of some patients dying with Alzheimer disease (Crapper et al., 1976, Brain, 99:67-80) and one case report has documented markedly elevated levels of manganese in the tissues of a patient with Alzheimer disease (Banta & Markesberg, 1977, Neurology, 27:213-216), which has led to the suggestion that high levels of these metals may be neurotoxic and lead to the development of Alzheimer disease. It was interesting that the aluminum ions were found to be associated mainly with the nuclear chromatin in brain regions most likely to display neurofibrillary tangles in Alzheimer disease. However, from a statistical point of view the absolute differences found for the aluminum levels between normal and Alzheimer brains were far from convincing. It has recently been suggested that defects in the transcriptional splicing of mRNA coding for the tau complex of microtubule associated proteins occur (for review see Kosik, 1990, Curr. Opinion Cell Biol., 2:101-104) and/or that inappropriate phosphorylation of these proteins exists (Grundke-Igbak et al., 1986, Proc. Natl. Acad. Sci. USA, 83:4913-4917; Wolozin & Davies, 1987, Ann. Neurol. 22:521-526; Hyman et al., 1988, Ann. Neurol., 23:371-379; Bancher et al., 1989, Brain Res., 477:90-99). Furthermore, reduction in the enzymes involved in the synthesis of acetylcholine has led to the view of Alzheimer disease as a cholinergic system failure (Danes & Moloney, 1976, Lancet, ii:1403-14). However, even if cholinergic neurons are most at risk in Alzheimer disease, it appears likely that these reductions in enzyme activity are secondary to the degenerative process itself rather than causally related.

At present, there are no agents that are consistently effective in preventing the progression of the disease. Acetylcholinesterase inhibitors are the mainstay of therapy. The majority of therapeutics that are in current use focus on the management of the symptoms of AD. These strategies have employed the use of anti-psychiatric drugs as well as neuroleptic agents and acetylcholinesterase inhibitors. However, due to the side effects and unattractive dosing requirements of these drugs, new methods and compounds that are able to treat AD and its symptoms are highly desirable.

2.3 Mild Cognitive Impairment

Mild cognitive impairment or minimal cognitive impairment (MCI) refers to a stage of cognitive impairment and specifically a subtype with memory loss prior to attaining clinical criteria for dementia in Alzheimer disease (AD). However, no completely reliable means, other than long-term follow-up and eventual autopsy, exist to distinguish between patients experiencing MCI due to preclinical AD and patients experiencing MCI due to less frequently occurring conditions (Petersen et al., Arch Neurol, 2001, 58(12): 1985-92). In this context, MCI is regarded as a high-risk condition that precedes AD in a large proportion of cases. The relatively recent formulation of MCI follows previous attempts to characterize cognitive decline associated with aging, including benign senescent forgetfulness, age-associated memory impairment, and age-associated cognitive decline (Crook et al., Dev Neuropsychol., 1986, 2: 261-276; Kral, CMAJ 1962, 86: 257-260; Levy et al., Int Psychogeriatr 1994, 6(1): 63-8). In contrast with many previous terms, individuals with MCI have a condition that is different from normal aging in that long-term follow-up indicates that they progress as a group to AD at an accelerated rate (Petersen et al., JAMA, 1995, 273(16): 1274-8; Petersen et al., Arch Neurol, 1999, 56(3): 303-8). Other terms with connotations similar to MCI include isolated memory impairment, incipient dementia, and dementia prodrome, although these latter terms are not nearly as widely accepted as MCI.

The pathophysiology of MCI is unknown. One hypothesis is that it often results from a gradual build-up of senile plaques and neurofibrillary tangles in areas of the cerebral cortex targeted by AD before the density of these lesions reaches the threshold necessary for the histopathologic diagnosis of AD. Similarly, the development of certain neurotransmitter deficiencies, and especially a cortical cholinergic deficiency, in the most common amnestic form of MCI is hypothesized. In the few studies undertaken to date, most patients with MCI have neuropathologic changes akin to AD, while a few clinically similar individuals do not have significant numbers of AD-like lesions (Mufson et al., Exp Neurol, 1999, 158(2): 469-90; Price et al., Ann Neurol, 1999, 45(3): 358-68; Troncoso et al., Neurobiol Aging, 1996, 17(3): 365-71).

MCI is a heterogeneous condition due to numerous different causes, which may overlap in individual patients. In an attempt to distinguish among patient groups, emphasis is often placed on whether memory is involved or single nonmemory domains are involved instead. The most common form of MCI is thought to be amnesic MCI, in which the single domain affected is memory. A large percentage of these patients progress to AD. A presumably less common form of MCI is one in which multiple cognitive domains are affected. This is at least theoretically associated with atypical variants of AD and dementia associated with cerebrovascular disease. A third postulated type is one in which a single nonmemory domain is affected. Such a condition is believed to evolve into frontotemporal dementia, Lewy body dementia, primary progressive aphasia, dementia in Parkinson disease, and other atypical variants of AD.

There is no treatment for MCI at present. Several trials are currently underway to determine whether cholinesterase inhibitors, anti-inflammatory agents, and antioxidants may be beneficial in MCI. Smaller scale studies suggest that at least cholinesterase inhibitors may improve the memory loss, although larger scale studies are necessary to ascertain this more rigorously. Freo et al., Soc Neurosci Abstr, 677, 2001.

2.4 Depression

Depression is characterized by feelings of intense sadness or pessimistic worry, agitation, self-deprecation, mental slowing, insomnia, anorexia, loss of drive, enthusiasm and libido. The influence of chronic antidepressant administration on expression of the three major phosphodiesterase (PDE) 4 subtypes found in brain (PDE4A, PDE4B, and PDE4C) was examined. Takahashi et al., The Journal of Neuroscience, 1999, 19(2):610-618. The treatments included representatives of four major classes of antidepressants such as selective reuptake inhibitors of serotonin (sertraline and fluoxetine), or norepinephrine (desipramine), a monoamine oxidase inhibitor (tranylcypromine), and electroconvulsive seizure. Id. The results of this study demonstrate that chronic antidepressant administration increased expression of PDE4A and PDE4B on cerebral cortex and expression of PDE4B in nucleus accumbens. Upregulation of PDE4A and PDE4B may represent a compensatory response to antidepressant treatment and activation of the cAMP system.

The antidepressant effects of Rolipram, a selective inhibitor of PDE4, in the central nervous system were studied in animal models and clinical trials. Zhu et al., CNS Drug Reviews, Vol. 7, No. 4, 387-398, 2001. It has been reported that PDE4 is responsible for hydrolysis of the cyclic nucleotide cAMP and cGMP, particularly in nerve and immune cells. Id. Rolipram induces elevation of intracellular cAMP, and increases synthesis and release of norepinephrine, which enhance central noradrenergic transmission. Id. Rolipram attenuates endogenous depression and inflammation in the central nervous system. Id. However, there are some discrepancies between in vitro and in vivo effects of Rolipram, as well as between results obtained in animal models and clinical studies. Id. In addition, the clinical use of Rolipram is limited due to its behavioral and other side effects. Therefore, there is a significant need for a selective PDE4 inhibitor with higher potency and lower toxicity.

2.5 Defective Long-Term Memory

Rubinstein-Taybi syndrome (RTS) is a human genetic disorder characterized by mental retardation and physical abnormalities including broad thumbs, big and broad toes, short stature, and craniofacial anomalies. Bourtchouladze et al., PNAS, 2003, vol. 100, no. 18. RTS occurs in about 1 in 125,000 births and accounts for as many as 1 in 300 cases of institutionalized mentally retarded people. Id. In many patients, RTS has been mapped to chromosome 16p13.3, a genomic region containing cAMP-responsive element binding protein (CREB)-binding protein (CBP). Id. Many RTS patients are heterozygous for CBP mutations that yield truncations of the CBP C terminus, suggesting that a dominant-negative mechanism may contribute to the clinical symptoms of defective long-term memory. Id.

The studies by Bourtchouladze et al. demonstrated that CREB and CBP likely function together as a molecular switch during long-term memory formation. Id. They demonstrated that PDE4 inhibitors Rolipram and HT0712 abolished the long-term memory defects of CBP^{+/-} mutant mice. Id. It was reported that the inhibitors of PDE4 enhanced CREB-dependent gene expression and ameliorated the long-term memory defects of CBP^{+/-} mutant mice in a dose-dependent manner. Id.

2.6 Selective Cytokine Inhibitory Drugs

Compounds referred to as SelCIDS™ (Celgene Corporation) or Selective Cytokine Inhibitory Drugs have been synthesized and tested. These compounds potently inhibit TNF-α production, but exhibit modest inhibitory effects on LPS induced IL1β and IL12, and do not inhibit IL6 even at high drug concentrations. In addition, SelCIDs™ tend to produce a modest IL10 stimulation. L. G. Corral, et al., Ann. Rheum. Dis. 58:(Suppl I) 1107-1113 (1999).

Further characterization of the selective cytokine inhibitory drugs shows that they are potent PDE4 inhibitors. PDE4 is one of the major phosphodiesterase isoenzymes found in human myeloid and lymphoid lineage cells. The enzyme plays a crucial part in regulating cellular activity by degrading the ubiquitous second messenger cAMP and maintaining it at low intracellular levels. Id. In the central nervous system (CNS), PDE4 is expressed in neurons of many portions of the brain, including dopaminergic neurons of the substantia nigra (Chemy and Davis, J Comp Neurol 407:287-301 1999), a key target area of damage in Parkinson disease, and in astrocytes, a cell type associated with inflammation in the brain. Elevation of cAMP in neuronal precursors also promotes secretion of norepinephrine and acetylcholine (Rabe et al., J Cyclic Nucleotide Res 8:371-384, 1982), neurite extension (Traynor and Schubert, Brain Res 316:197-204, 1984; Westlund et al., Int J Dev Neurosci 10:361-373, 1992), and serotonin signaling (Akaike et al. Brain Res 620:58-6, 1993), and drives differentiation of dopaminergic neurons from embryonic stem cells (Iacovitti et al., Brain Res 912:99-104, 2001). Inhibition of PDE4 activity results in increased cAMP levels leading to the modulation of LPS induced cytokines including inhibition of TNF-α production in monocytes as well as in lymphocytes.

### 3. SUMMARY OF THE INVENTION

This invention encompasses methods of treating or preventing central nervous system disorders and related disorders which comprise administering to a patient in need of such treatment or prevention a therapeutically or prophylactically effective amount of a selective cytokine inhibitory drug, or a pharmaceutically acceptable salt, solvate, hydrate, stereoisomer, clathrate, or prodrug thereof. Central nervous system disorders include, but are not limited to, Alzheimer disease, mild cognitive impairment (MCI), Parkinson disease, depression, defective long-term memory, Huntington disease, multiple Sclerosis, delerium, or disturbances in consciousness that occur over a short period (>f time, and amnestic disorder, or discreet memory impairments that occur in the absence of other central nervous system impairments. The invention also encompasses methods of managing central nervous system disorders (e.g., lengthening the time of remission of their symptoms) which comprise administering to a patient in need of such management a prophylactically effective amount of a selective cytokine inhibitory drug, or a pharmaceutically acceptable salt, solvate, hydrate, stereoisomer, clathrate, or prodrug thereof. Each of these methods includes specific dosing or dosing regimens including cycling therapy.

The invention further encompasses pharmaceutical compositions, single unit dosage forms, and kits suitable for use in treating, preventing and/or managing central nervous system disorders, which comprise one or more selective cytokine inhibitory drugs, or a pharmaceutically acceptable salt, solvate, hydrate, stereoisomer, clathrate, or prodrug thereof.

The selective cytokine inhibitory drugs, or compounds of the invention, which are described in detail below, are small organic molecules, i.e., having a molecule weight less than 1,000 g/mol. The compounds preferably inhibit PDE4 activity and TNF-α.

In particular embodiments of the invention, a selective cytokine inhibitory drug is used, administered, or formulated with one or more second active ingredients to treat, prevent or manage central nervous system disorders. Examples of the second active ingredients include but are not limited to dopamine agonists, Levodopa, compounds used to augment Levodopa therapy such as monoamine oxidase inhibitors (MAO) and catechol-O-methyltransferase inhibitors (COMT), amantadine, anticholinergics, antiemetics, and other standard therapies for central nervous system disorders. In another example, the second active ingredients are anti-inflammatory agents, including, but not limited to, nonsteroidal anti-inflammatory drugs (NSAIDs), Methotrexate, Leflunomide, antimalarial drugs and sulfasalazine, gold salts, glucocorticoids, immunosuppresive agents, and other standard therapies for central nervous system disorders.

### 4. DETAILED DESCRIPTION OF THE INVENTION

A first embodiment of the invention encompasses methods of treating or preventing a central nervous system disorder, which comprises administering to a patient in need of such treatment or prevention a therapeutically or prophylactically effective amount of a selective cytokine inhibitory drug, or a pharmaceutically acceptable salt, solvate, hydrate, stereoisomer, clathrate, or prodrug thereof. Central nervous system disorders, include, but are not limited to, Parkinson disease; bradykinesia; muscle rigidity; parkinsonian tremor; parkinsonian gait; motion freezing; depression; defective long-term memory, Rubinstein-Taybi syndrome (RTS); dementia; sleep disorders; postural instability; hypokinetic disorders; inflammation; synuclein disorders; multiple system artrophies; striatonigral degeneration; olivopontocerebellar atrophy; Shy-Drager syndrome; motor neuron disease with parkinsonian features; Lewy body dementia; Tau pathology disorders; progressive supranculear palsy; corticobasal degeneration; frontotemporal dementia; amyloid pathology disorders; mild cognitive impairment; Alzheimer disease; Alzheimer disease with parkinsonism; genetic disorders that can have parkinsonian features; Wilson disease; Hallervorden-Spatz disease; Chediak-Hagashi disease; SCA-3 spinocerebellar ataxia; X-linked dystonia parkinsonism; Huntington disease; prion disease; hyperkinetic disorders; chorea; ballismus; dystonia tremors; Amyotrophic Lateral Sclerosis (ALS); CNS trauma and myoclonus.

Another embodiment of the invention encompasses methods of managing a central nervous system disorder, which comprises administering to a patient in need of such management a prophylactically effective amount of a selective cytokine inhibitory drug, or a pharmaceutically acceptable salt, solvate, hydrate, stereoisomer, clathrate, or prodrug thereof.

Another embodiment of the invention encompasses a method of treating, preventing and/or managing a central nervous system disorder, which comprises administering to a patient in need of such treatment, prevention and/or management a therapeutically or prophylactically effective amount of a selective cytokine inhibitory drug, or a pharmaceutically acceptable salt, solvate, hydrate, stereoisomer, clathrate, or prodrug thereof, and a therapeutically or prophylactically effective amount of a second active agent. Without being limited by theory, it is believed that certain selective cytokine inhibitory drugs and agents conventionally used in central nervous system disorders can act in complementary or synergistic ways in the treatment or management of the disorders. It is also believed that the combined use of such agents may reduce or eliminate adverse effects associated with some selective cytokine inhibitory drugs, thereby allowing the administration of larger amounts of selective cytokine inhibitory drugs to patients and/or increasing patient compliance. It is further believed that some selective cytokine inhibitory drugs may reduce or eliminate adverse effects associated with some conventional agents, thereby allowing the administration of larger amounts of the agents to patients and/or increasing patient compliance.

Another embodiment of the invention encompasses a method of reversing, reducing or avoiding an adverse effect associated with the administration of conventional therapy for central nervous system disorders to a patient suffering from central nervous system disorders or a related disorder, which comprises administering to a patient in need of such reversion, reduction or avoidance a therapeutically or prophylactically effective amount of a selective cytokine inhibitory drug, or a pharmaceutically acceptable salt, solvate, hydrate, stereoisomer, clathrate, or prodrug thereof.

Yet another embodiment of the invention encompasses a pharmaceutical composition comprising a selective cytokine inhibitory drug, or a pharmaceutically acceptable salt, solvate, hydrate, stereoisomer, clathrate, or prodrug thereof, and a pharmaceutically acceptable carrier, diluent or excipient wherein the composition is adapted for parenteral, oral or transdermal administration and the amount is sufficient to treat or prevent a central nervous system disorder, or to ameliorate the symptoms or progress of the disorder.

Also encompassed by the invention are single unit dosage forms comprising a selective cytokine inhibitory drug, or a pharmaceutically acceptable salt, solvate, hydrate, stereoisomer, clathrate, or prodrug thereof.

Second active agents can be large molecules (e.g., proteins) or small molecules (e.g., synthetic inorganic, organometallic, or organic molecules). The examples of the second active agent include, but are not limited to, cytokines, hematopoietic growth factors, anti-cancer agents such as topoisomerase inhibitors, anti-angiogenic agents, microtubule stabilizing agents, alkylating agents; acetylcholinesterase inhibitors; antivirals; antifungals; antibiotics; anti-inflammatories; immunomodulatory agents; immunosuppressive agents such as cyclosporins; and other known or conventional agents used in patients with central nervous system disorders. Specific second active agents include but are not limited to a dopamine agonist or antagonist for Parkinson disease or an acetylcholinesterate inhibitor for Alzheimer disease.

The invention also encompasses kits which comprise a selective cytokine inhibitory drug, or a pharmaceutically acceptable salt, solvate, hydrate, stereoisomer, clathrate, or prodrug thereof, a second active ingredient.

4.1 Selective Cytokine Inhibitory Drugs

Compounds used in the invention include racemic, stereomerically pure and stereomerically enriched selective cytokine inhibitory drugs, stereomerically and enantiomerically pure compounds that have selective cytokine inhibitory activities, and pharmaceutically acceptable salts, solvates, hydrates, stereoisomers, clathrates, and prodrugs thereof. Preferred compounds used in the invention are known Selective Cytokine Inhibitory Drugs (SelCIDS™) of Celgene Corporation, NJ.

As used herein and unless otherwise indicated, the terms “selective cytokine inhibitory drugs” and “SelCIDs™” encompass small molecule drugs, e.g., small organic molecules which are not peptides, proteins, nucleic acids, oligosaccharides or other macromolecules. Preferred compounds inhibit TNF-α production. Compounds may also have a modest inhibitory effect on LPS induced IL1β and IL12. More preferably, the compounds of the invention are potent PDE4 inhibitors.

Specific examples of selective cytokine inhibitory drugs include, but are not limited to, the cyclic imides disclosed in U.S. Pat. Nos. 5,605,914 and 5,463,063; the cycloalkyl amides and cycloalkyl nitrites of U.S. Pat. Nos. 5,728,844, 5,728,845, 5,968,945, 6,180,644 and 6,518,281; the aryl amides (for example, an embodiment being N-benzoyl-3-amino-3-(3′,4′-dimethoxyphenyl)-propanamide) of U.S. Pat. Nos. 5,801,195, 5,736,570, 6,046,221 and 6,284,780; the imide/amide ethers and alcohols (for example, 3-phthalimido-3-(3′,4′-dimethoxyphenyl)propan-1-ol) disclosed in U.S. Pat. No. 5,703,098; the succinimides and maleimides (for example methyl 3-(3′,4′,5′6′-petrahydrophthalimdo)-3-(3″,4″-dimethoxyphenyl)propionate) disclosed in U.S. Pat. No. 5,658,940; imido and amido substituted alkanohydroxamic acids disclosed in U.S. Pat. No. 6,214,857 and WO 99/06041; substituted phenethylsulfones disclosed in U.S. Pat. Nos. 6,011,050 and 6,020,358; fluoroalkoxy-substituted 1,3-dihydro-isoindolyl compounds disclosed in U.S. patent application Ser. No. 10/748,085 filed on Dec. 29, 2003; substituted imides (for example, 2-phthalimido-3-(3′,4′-dimethoxyphenyl)propane) disclosed in U.S. Pat. No. 6,429,221; substituted 1,3,4-oxadiazoles (for example, 2-[1-(3-cyclopentyloxy-4-methoxyphenyl)-2-(1,3,4-oxadiazole-2-yl)ethyl]-5-methylisoindoline-1,3-dione) disclosed in U.S. Pat. No. 6,326,388; cyano and carboxy derivatives of substituted styrenes (for example, 3,3-bis-(3,4-dimethoxyphenyl)acrylonitrile) disclosed in U.S. Pat. Nos. 5,929,117, 6,130,226, 6,262,101 and 6,479,554; isoindoline-1-one and isoindoline-1,3-dione substituted in the 2-position with an α-(3,4-disubstituted phenyl)alkyl group and in the 4- and/or 5-position with a nitrogen-containing group disclosed in WO 01/34606 and U.S. Pat. No. 6,667,316; and imido and amido substituted acylhydroxamic acids (for example, (3-(1,3-dioxoisoindoline-2-yl)-3-(3-ethoxy-4-methoxyphenyl)propanoylamino)propanoate disclosed in WO 01/45702 and U.S. Pat. No. 6,699,899. Other selective cytokine inhibitory drugs include diphenylethylene compounds disclosed in U.S. provisional application No. 60/452,460, filed Mar. 5, 2003, the contents of which are incorporated by reference herein in their entirety. Other selective cytokine inhibitory drugs include isoindoline compounds disclosed in U.S. patent application Ser. Nos. 10/900,332 and 10/900,270, both filed on Jul. 28, 2004. The entireties of each of the patents and patent applications identified herein are incorporated herein by reference.

Additional selective cytokine inhibitory drugs belong to a family of synthesized chemical compounds of which typical embodiments include 3-(1,3-dioxobenzo-[f]isoindol-2-yl)-3-(3-cyclopentyloxy-4-methoxyphenyl)propionamide and 3-(1,3-dioxo-4-azaisoindol-2-yl)-3-(3,4-dimethoxyphenyl)-propionamide.

Other specific selective cytokine inhibitory drugs belong to a class of non-polypeptide cyclic amides disclosed in U.S. Pat. Nos. 5,698,579, 5,877,200, 6,075,041 and 6,200,987, and WO 95/01348, each of which is incorporated herein by reference. Representative cyclic amides include compounds of the formula:

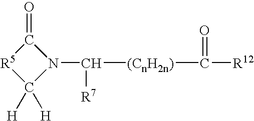

wherein n has a value of 1, 2, or 3;

R⁵ is o-phenylene, unsubstituted or substituted with 1 to 4 substitutents each selected independently from the group consisting of nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkyl of 1 to 10 carbon atoms, and halo;

R⁷ is (i) phenyl or phenyl substituted with one or more substitutents each selected independently of the other from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo, (ii) benzyl unsubstituted or substituted with 1 to 3 substitutents selected from the group consisting of nitro, cyano, trifluoromethyl, carbothoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo, (iii) naphthyl, and (iv) benzyloxy;

R¹² is —OH, alkoxy of 1 to 12 carbon atoms, or

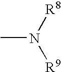

R⁸ is hydrogen or alkyl of 1 to 10 carbon atoms; and

R⁹ is hydrogen, alkyl of 1 to 10 carbon atoms, —COR¹⁰, or —SO₂R¹⁰, wherein R¹⁰ is hydrogen, alkyl of 1 to 10 carbon atoms, or phenyl.

Specific compounds of this class include, but are not limited to:
3-phenyl-2-(1-oxoisoindolin-2-yl)propionic acid;
3-phenyl-2-(1-oxoisoindolin-2-yl)propionamide;
3-phenyl-3-(1-oxoisoindolin-2-yl)propionic acid;
3-phenyl-3-(1-oxoisoindolin-2-yl)propionamide;
3-(4-methoxyphenyl)-3-(1-oxisoindolin-yl)propionic acid;
3-(4-methoxyphenyl)-3-(1-oxisoindolin-yl)propionamide;
3-(3,4-dimethoxyphenyl)-3-(1-oxisoindolin-2-yl)propionic acid;
3-(3,4-dimethoxy-phenyl)-3-(1-oxo-1,3-dihydroisoindol-2-yl)propionamide;
3-(3,4-dimethoxyphenyl)-3-(1-oxisoindolin-2-yl)propionamide;
3-(3,4-diethoxyphenyl)-3-(1-oxoisoindolin-yl)propionic acid;
methyl 3-(1-oxoisoindolin-2-yl)-3-(3-ethoxy-4-methoxyphenyl)propionate;
3-(1-oxoisoindolin-2-yl)-3-(3-ethoxy-4-methoxyphenyl)propionic acid;
3-(1-oxoisoindolin-2-yl)-3-(3-propoxy-4-methoxyphenyl)propionic acid;
3-(1-oxoisoindolin-2-yl)-3-(3-butoxy-4-methoxyphenyl)propionic acid;
3-(1-oxoisoindolin-2-yl)-3-(3-propoxy-4-methoxyphenyl)propionamide;
3-(1-oxoisoindolin-2-yl)-3-(3-butoxy-4-methoxyphenyl)propionamide;
methyl 3-(1-oxoisoindolin-2-yl)-3-(3-butoxy-4-methoxyphenyl)propionate; and
methyl 3-(1-oxoisoindolin-2-yl)-3-(3-propoxy-4-methoxyphenyl)propionate.

Other representative cyclic amides include compounds of the formula:

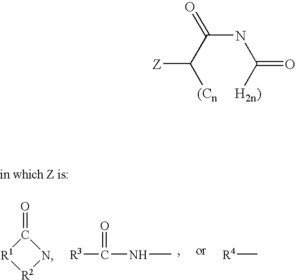

in which:

R¹ is the divalent residue of (i) 3,4-pyridine, (ii) pyrrolidine, (iii) imidizole, (iv) naphthalene, (v) thiophene, or (vi) a straight or branched alkane of 2 to 6 carbon atoms, unsubstituted or substituted with phenyl or phenyl substituted with nitro, cyano, trifluoromethyl, carbethoxy, carbomethoxy, carbopropoxy, acetyl, carbamyl, acetoxy, carboxy, hydroxy, amino, alkyl of 1 to 10 carbon atoms, alkoxy of 1 to 10 carbon atoms, or halo, wherein the divalent bonds of said residue are on vicinal ring carbon atoms;

R² is —CO— or —SO₂—;

R³ is (i) phenyl substituted with 1 to 3 substitutents each selected independently from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo, (ii) pyridyl, (iii) pyrrolyl, (iv) imidazolyl, (iv) naphthyl, (vi) thienyl, (vii) quinolyl, (viii) furyl, or (ix) indolyl;

R⁴ is alanyl, arginyl, glycyl, phenylglycyl, histidyl, leucyl, isoleucyl, lysyl, methionyl, prolyl, sarcosyl, seryl, homoseryl, threonyl, thyronyl, tyrosyl, valyl, benzimidol-2-yl, benzoxazol-2-yl, phenylsulfonyl, methylphenylsulfonyl, or phenylcarbamoyl; and

n has a value of 1, 2, or 3. Other representative cyclic amides include compounds of the formula:

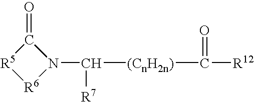

in which R⁵ is (i) o-phenylene, unsubstituted or substituted with 1 to 4 substitutents each selected independently from nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkoxy of 1 to 10 carbon atoms, or halo, or (ii) the divalent residue of pyridine, pyrrolidine, imidizole, naphthalene, or thiophene, wherein the divalent bonds are on vicinal ring carbon atoms;

R⁶ is —CO —, —CH₂—, or —SO₂—;

R⁷ is (i) hydrogen if R⁶ is —SO₂—, (ii) straight, branched, or cyclic alkyl of 1 to 12 carbon atoms, (iii) pyridyl, (iv) phenyl or phenyl substituted with one or more substitutents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo, (v) alkyl of 1 to 10 carbon atoms, (vi) benzyl unsubstituted or substituted with 1 to 3 substitutents selected from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo, (vii) naphthyl, (viii) benzyloxy, or (ix) imidazol-4-yl methyl;

R¹² is —OH, alkoxy of 1 to 12 carbon atoms, or

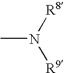

n has a value of 0, 1, 2, or 3;

R^{8′} is hydrogen or alkyl of 1 to 10 carbon atoms; and

R^{9′} is hydrogen, alkyl of 1 to 10 carbon atoms, —COR¹⁰, or —SO₂R¹⁰ in which R¹⁰ is hydrogen, alkyl of 1 to 10 carbon atoms, or phenyl.

Other representative imides include compounds of the formula:

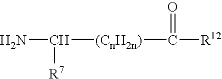

in which R⁷ is (i) straight, branched, or cyclic alkyl of 1 to 12 carbon atoms, (ii) pyridyl, (iii) phenyl or phenyl substituted with one or more substitutents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo, (iv) benzyl unsubstituted or substituted with one to three substitutents selected from the group consisting of nitro, cyano, trifluoromethyl, carbethoxy, carbomethoxy, carbopropoxy, acetyl, carbamoyl, acetoxy, carboxy, hydroxy, amino, alkyl of 1 to 4 carbon atoms, alkoxy of 1 to 4 carbon atoms, or halo, (v) naphthyl, (vi) benzyloxy, or (vii) imidazol-4-ylmethyl;

R¹² is —OH, alkoxy of 1 to 12 carbon atoms, —O—CH₂-pyridyl, —O-benzyl or

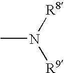

where n has a value of 0, 1, 2, or 3;

R^{8′} is hydrogen or alkyl of 1 to 10 carbon atoms; and

R^{9′} is hydrogen, alkyl of 1 to 10 carbon atoms, —CH₂-pyridyl, benzyl, —COR¹⁰, or —SO₂R¹⁰ in which R¹⁰ is hydrogen, alkyl of 1 to 4 carbon atoms, or phenyl.

Other specific selective cytokine inhibitory drugs include the imido and amido substituted alkanohydroxamic acids disclosed in WO 99/06041 and U.S. Pat. No. 6,214,857, each of which is incorporated herein by reference. Examples of such compound include, but are not limited to:

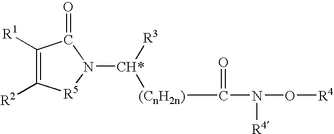

wherein each of R¹ and R², when taken independently of each other, is hydrogen, lower alkyl, or R¹ and R², when taken together with the depicted carbon atoms to which each is bound, is o-phenylene, o-naphthylene, or cyclohexene-1,2-diyl, unsubstituted or substituted with 1 to 4 substitutents each selected independently from the group consisting of nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkoxy of 1 to 10 carbon atoms, and halo;

R³ is phenyl substituted with from one to four substitutents selected from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, alkylthio of 1 to 10 carbon atoms, benzyloxy, cycloalkoxy of 3 to 6 carbon atoms, C₄-C₆-cycloalkylidenemethyl, C₃-C₁₀-alkylidenemethyl, indanyloxy, and halo;

R⁴ is hydrogen, alkyl of 1 to 6 carbon atoms, phenyl, or benzyl;

R^{4′} is hydrogen or alkyl of 1 to 6 carbon atoms;

R⁵ is —CH₂—, —CH₂—CO—, —SO₂—, —S—, or —NHCO—; and

n has a value of 0, 1, or 2; and

the acid addition salts of said compounds which contain a nitrogen atom capable of being protonated.

Additional specific selective cytokine inhibitory drugs used in the invention include, but are not limited to:
3-(3-ethoxy-4-methoxyphenyl)-N-hydroxy-3-(1-oxoisoindolinyl)propionamide;
3-(3-ethoxy-4-methoxyphenyl)-N-methoxy-3-(1-oxoisoindolinyl)propionamide;
N-benzyloxy-3-(3-ethoxy-4-methoxyphenyl)-3-phthalimidopropionamide;
N-benzyloxy-3-(3-ethoxy-4-methoxyphenyl)-3-(3-nitrophthalimido)propionamide;
N-benzyloxy-3-(3-ethoxy-4-methoxyphenyl)-3-(1-oxoisoindolinyl)propionamide;
3-(3-ethoxy-4-methoxyphenyl)-N-hydroxy-3-phthalimidopropionamide;
N-hydroxy-3-(3,4-dimethoxyphenyl)-3-phthalimidopropionamide;
3-(3-ethoxy-4-methoxyphenyl)-N-hydroxy-3-(3-nitrophthalimido)propionamide;
N-hydroxy-3-(3,4-dimethoxyphenyl)-3-(1-oxoisoindolinyl)propionamide;
3-(3-ethoxy-4-methoxyphenyl)-N-hydroxy-3-(4-methyl-phthalimido)propionamide;
3-(3-cyclopentyloxy-4-methoxyphenyl)-N-hydroxy-3-phthalimidopropionamide;
3-(3-ethoxy-4-methoxyphenyl)-N-hydroxy-3-(1,3-dioxo-2,3-dihydro-1H-benzo[f]isoindol-2-yl)propionamide;
N-hydroxy-3-{3-(2-propoxy)-4-methoxyphenyl}-3-phthalimidopropionamide;
3-(3-ethoxy-4-methoxyphenyl)-3-(3,6-difluorophthalimido)-N-hydroxypropionamide;
3-(4-aminophthalimido)-3-(3-ethoxy-4-methoxyphenyl)-N-hydroxypropionamide;
3-(3-aminophthalimido)-3-(3-ethoxy-4-methoxyphenyl)-N-hydroxypropionamide;
N-hydroxy-3-(3,4-dimethoxyphenyl)-3-(1-oxoisoindolinyl)propionamide;
3-(3-cyclopentyloxy-4-methoxyphenyl)-N-hydroxy-3-(1-oxoisoindolinyl) propionamide; and
N-benzyloxy-3-(3-ethoxy-4-methoxyphenyl)-3-(3-nitrophthalimido)propionamide.

Additional selective cytokine inhibitory drugs used in the invention include the substituted phenethylsulfones substituted on the phenyl group with a oxoisoindine group. Examples of such compounds include, but are not limited to, those disclosed in U.S. Pat. No. 6,020,358, which is incorporated herein by reference, which include the following:

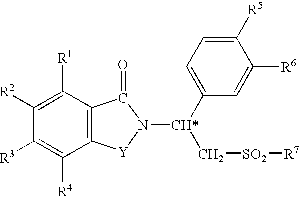

wherein the carbon atom designated * constitutes a center of chirality;

Y is C═O, CH₂, SO₂, or CH₂C═O; each of R¹, R², R³, and R⁴, independently of the others, is hydrogen, halo, alkyl of 1 to 4 carbon atoms, alkoxy of 1 to 4 carbon atoms, nitro, cyano, hydroxy, or —NR⁸R⁹; or any two of R¹, R², R³, and R⁴ on adjacent carbon atoms, together with the depicted phenylene ring are naphthylidene;

each of R⁵ and R⁶, independently of the other, is hydrogen, alkyl of 1 to 4 carbon atoms, alkoxy of 1 to 4 carbon atoms, cyano, or cycloalkoxy of up to 18 carbon atoms;

R⁷ is hydroxy, alkyl of 1 to 8 carbon atoms, phenyl, benzyl, or NR^{8′}R^{9′};

each of R⁸ and R⁹ taken independently of the other is hydrogen, alkyl of 1 to 8 carbon atoms, phenyl, or benzyl, or one of R⁸ and R⁹ is hydrogen and the other is —COR¹⁰ or —SO₂R¹⁰, or R⁸ and R⁹ taken together are tetramethylene, pentamethylene, hexamethylene, or —CH₂CH₂X¹CH₂CH₂— in which X¹ is —O—, —S— or —NH—; and

each of R^{8′} and R^{9′} taken independently of the other is hydrogen, alkyl of 1 to 8 carbon atoms, phenyl, or benzyl, or one of R^{8′} and R^{9′} is hydrogen and the other is —COR^{10′} or —SO₂R^{10′}, or R^{8′} and R^{9′} taken together are tetramethylene, pentamethylene, hexamethylene, or —CH₂CH₂X²CH₂CH₂— in which X² is —O—, —S—, or —NH—.

It will be appreciated that while for convenience the above compounds are identified as phenethylsulfones, they include sulfonamides when R⁷ is NR^{8′}R^{9′}.

Specific groups of such compounds are those in which Y is C═O or CH₂.

A further specific group of such compounds are those in which each of R¹, R², R³, and R⁴ independently of the others, is hydrogen, halo, methyl, ethyl, methoxy, ethoxy, nitro, cyano, hydroxy, or —NR⁸R⁹ in which each of R⁸ and R⁹ taken independently of the other is hydrogen or methyl or one of R⁸ and R⁹ is hydrogen and the other is —COCH₃.

Particular compounds are those in which one of R¹, R², R³, and R⁴ is —NH₂ and the remaining of R¹, R², R³, and R⁴ are hydrogen.

Particular compounds are those in which one of R¹, R², R³, and R⁴ is —NHCOCH₃ and the remaining of R¹, R², R³, and R⁴ are hydrogen.

Particular compounds are those in which one of R¹, R², R³, and R⁴ is —N(CH₃)₂ and the remaining of R¹, R², R³, and R⁴ are hydrogen.

A further preferred group of such compounds are those in which one of R¹, R², R³, and R⁴ is methyl and the remaining of R¹, R², R³, and R⁴ are hydrogen.

Particular compounds are those in which one of R¹, R², R³, and R⁴ is fluoro and the remaining of R¹, R², R³, and R⁴ are hydrogen.

Particular compounds are those in which each of R⁵ and R⁶, independently of the other, is hydrogen, methyl, ethyl, propyl, methoxy, ethoxy, propoxy, cyclopentoxy, or cyclohexoxy.

Particular compounds are those in which R⁵ is methoxy and R⁵ is monocycloalkoxy, polycycloalkoxy, and benzocycloalkoxy.

Particular compounds are those in which R⁵ is methoxy and R⁶ is ethoxy.

Particular compounds are those in which R⁷ is hydroxy, methyl, ethyl, phenyl, benzyl, or NR^{8′}R^{9′} in which each of R^{8′} and R^{9′} taken independently of the other is hydrogen or methyl.

Particular compounds are those in which R⁷ is methyl, ethyl, phenyl, benzyl or NR^{8′}R⁹ in which each of R^{8′} and R^{9′} taken independently of the other is hydrogen or methyl.

Particular compounds are those in which R⁷ is methyl.

Particular compounds are those in which R⁷ is NR^{8′}R^{9′} in which each of R^{8′} and R^{9′} taken independently of the other is hydrogen or methyl.

Additional selective cytokine inhibitory drugs include fluoroalkoxy-substituted 1,3-dihydro-isoindolyl compounds disclosed in U.S. patent application Ser. No. 10/748,085 filed on Dec. 29, 2003, which is incorporated herein by reference. Representative compounds are of formula:

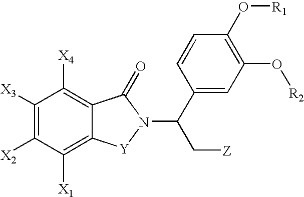

wherein:

Y is —C(O)—, —CH₂, —CH₂C(O)—, —C(O)CH₂—, or SO₂;

Z is —H, —C(O)R³, —(C₀₋₁-alkyl)-SO₂—(C₁₋₄-alkyl), —C₁₋₈-alkyl, —CH₂OH, CH₂(O)(C₁₋₈-alkyl) or —CN;

R₁ and R₂ are each independently —CHF₂, —C₁₋₈-alkyl, —C₃₋₁₈-cycloalkyl, or —(C₁₋₁₀-alkyl)(C₃₋₁₈-cycloalkyl), and at least one of R₁ and R₂ is CHF₂;

R³ is —NR⁴R⁵, -alkyl, —OH, —O-alkyl, phenyl, benzyl, substituted phenyl, or substituted benzyl;

R⁴ and R⁵ are each independently —H, —C₁₋₈-alkyl, —OH, —OC(O)R⁶;

R⁶ is —C₁₋₈-alkyl, -amino(C₁₋₈-alkyl), -phenyl, -benzyl, or -aryl;

X₁, X₂, X₃, and X₄ are each independently —H, -halogen, -nitro, —NH₂, —CF₃, —C₁₋₆-alkyl, —(C₀₋₄-alkyl)-(C₃₋₆-cycloalkyl), (C₀₋₄-alkyl)-NR⁷R⁸, (C₀₋₄-alkyl)-N(H)C(O)—(R⁸), (C₀₋₄-alkyl)-N(H)C(O)N(R⁷R⁸), (C₀₋₄-alkyl)-N(H)C(O)O(R⁷R⁸), (C₀₋₄-alkyl)-OR⁸, (C₀₋₄-alkyl)-imidazolyl, (C₀₋₄-alkyl)-pyrrolyl, (C₀₋₄-alkyl)-oxadiazolyl, or (C₀₋₄-alkyl)-triazolyl, or two of X₁, X₂, X₃, and X₄ may be joined together to form a cycloalkyl or heterocycloalkyl ring, (e.g., X₁ and X₂, X₂ and X₃, X₃ and X₄, X₁ and X₃, X₂ and X₄, or X₁ and X₄ may form a 3, 4, 5, 6, or 7 membered ring which may be aromatic, thereby forming a bicyclic system with the isoindolyl ring); and

R⁷ and R⁸ are each independently H, C₁₋₉-alkyl, C₃₋₆-cycloalkyl, (C₁₋₆-alkyl)-(C₃₋₆-cycloalkyl), (C₁₋₆-alkyl)-N(R⁷R⁸), (C₁₋₆-alkyl)-OR⁸, phenyl, benzyl, or aryl; or a pharmaceutically acceptable salt, solvate, hydrate, stereoisomer, clathrate, or prodrug thereof.

Additional selective cytokine inhibitory drugs include the enantiomerically pure compounds disclosed in U.S. patent application Ser. No. 10/392,195 filed on Mar. 19, 2003; international patent application nos. PCT/US03/08737 and PCT/US03/08738, filed on Mar. 20, 2003; U.S. provisional patent application Nos. 60/438,450 and 60/438,448 to G. Muller et al., both of which were filed on Jan. 7, 2003; U.S. provisional patent application No. 60/452,460 to G. Muller et al. filed on Mar. 5, 2003; and U.S. patent application Ser. No. 10/715,184 filed on Nov. 17, 2003, all of which are incorporated herein by reference. Preferred compounds include an enantiomer of 2-[1-(3-ethoxy-4-methoxyphenyl)-2-methylsulfonylethyl]-4-acetylaminoisoindoline-1,3-dione and an enantiomer of 3-(3,4-dimethoxy-phenyl)-3-(1-oxo-1,3-dihydro-isoindol-2-yl)-propionamide.

Preferred selective cytokine inhibitory drugs used in the invention are 3-(3,4-dimethoxy-phenyl)-3-(1-oxo-1,3-dihydro-isoindol-2-yl)-propionamide and cyclopropanecarboxylic acid {2-[1-(3-ethoxy-4-methoxy-phenyl)-2-methanesulfonyl-ethyl]-3-oxo-2,3-dihydro-1H-isoindol-4-yl}-amide, which are available from Celgene Corp., Warren, N.J. 3-(3,4-Dimethoxy-phenyl)-3-(1-oxo-1,3-dihydro-isoindol-2-yl)-propionamide has the following chemical structure:

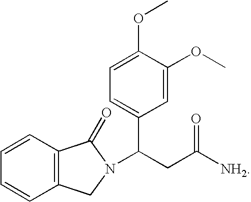

Other specific selective cytokine inhibitory drugs include, but are not limited to, the cycloalkyl amides and cycloalkyl nitriles of U.S. Pat. Nos. 5,728,844, 5,728,845, 5,968,945, 6,180,644 and 6,518,281, and WO 97/08143 and WO 97/23457, each of which is incorporated herein by reference. Representative compounds are of formula:

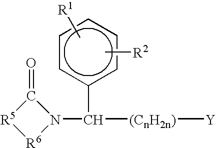

wherein:

one of R¹ and R² is R³—X— and the other is hydrogen, nitro, cyano, trifluoromethyl, carbo(lower)alkoxy, acetyl, carbamoyl, acetoxy, carboxy, hydroxy, amino, lower alkyl, lower alkoxy, halo, or R³—X—;

R³ is monocycloalkyl, bicycloalkyl, or benzocycloalkyl of up to 18 carbon atoms;

X is a carbon-carbon bond, —CH₂—, or —O—;

R⁵ is (i) o-phenylene, unsubstituted or substituted with 1 to 3 substitutents each selected independently from nitro, cyano, halo, trifluoromethyl, carbo(lower)alkoxy, acetyl, or carbamoyl, unsubstituted or substituted with lower alkyl, acetoxy, carboxy, hydroxy, amino, lower alkylamino, lower acylamino, or lower alkoxy; (ii) a vicinally divalent residue of pyridine, pyrrolidine, imidazole, naphthalene, or thiophene, wherein the divalent bonds are on vicinal ring carbon atoms; (iii) a vicinally divalent cycloalkyl or cycloalkenyl of 4-10 carbon atoms, unsubstituted or substituted with 1 to 3 substitutents each selected independently from the group consisting of nitro, cyano, halo, trifluoromethyl, carbo(lower)alkoxy, acetyl, carbamoyl, acetoxy, carboxy, hydroxy, amino, lower alkylamino, lower alkyl, lower alkoxy, or phenyl; (iv) vinylene di-substituted with lower alkyl; or (v) ethylene, unsubstituted or monosubstituted or disubstituted with lower alkyl;

R⁶ is —CO—, —CH₂—, or —CH₂CO—;

Y is —COZ, —C≡N, —OR⁸, lower alkyl, or aryl;

Z is —NH₂, —OH, —NHR, —R⁹, or —OR⁹

R⁸ is hydrogen or lower alkyl;

R⁹ is lower alkyl or benzyl; and,

n has a value of 0, 1, 2, or 3.

In another embodiment, one of R¹ and R² is R³—X— and the other is hydrogen, nitro, cyano, trifluoromethyl, carbo(lower)alkoxy, acetyl, carbamoyl, acetoxy, carboxy, hydroxy, amino, lower alkyl, lower alkoxy, halo, or R³—X—;

R³ is monocycloalkyl of up to 10 carbon atoms, polycycloalkyl of up to 10 carbon atoms, or benzocyclic alkyl of up to 10 carbon atoms;

X is —CH₂—, or —O—;

R⁵ is (i) the vicinally divalent residue of pyridine, pyrrolidine, imidazole, naphthalene, or thiophene, wherein the two bonds of the divalent residue are on vicinal ring carbon atoms;

(ii) a vicinally divalent cycloalkyl of 4-10 carbon atoms, unsubstituted or substituted with 1 to 3 substitutents each selected independently from the group consisting of nitro, cyano, halo, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, or phenyl;

(iii) di-substituted vinylene, substituted with nitro, cyano, trifluoromethyl, carbethoxy, carbomethoxy, carbopropoxy, acetyl, carbamoyl, carbamoyl substituted with and alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, or halo;

(iv) ethylene, unsubstituted or substituted with 1 to 2 substitutents each selected independently from nitro, cyano, trifluoromethyl, carbethoxy, carbomethoxy, carbopropoxy, acetyl, carbamoyl, carbamoyl substituted with and alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, or halo;

R⁶ is —CO—, —CH₂—, or —CH₂CO—;

Y is —COX, —C≡N, —OR⁸, alkyl of 1 to 5 carbon atoms, or aryl;

X is —NH₂, —OH, —NHR, —R⁹, —OR⁹, or alkyl of 1 to 5 carbon atoms;

R⁸ is hydrogen or lower alkyl;

R⁹ is alkyl or benzyl; and,

n has a value of 0, 1, 2, or 3.

In another embodiment, one of R¹ and R² is R³—X— and the other is hydrogen, nitro, cyano, trifluoromethyl, carbo(lower)alkoxy, acetyl, carbamoyl, acetoxy, carboxy, hydroxy, amino, lower alkyl, lower alkoxy, halo, HF₂CO, F₃CO, or R³—X—;

R³ is monocycloalkyl, bicycloalkyl, benzocyclo alkyl of up to 18 carbon atoms, tetrahydropyran, or tetrahydrofuran;

X is a carbon-carbon bond, —CH₂—, —O—, or —N═;

R⁵ is (i) o-phenylene, unsubstituted or substituted with 1 to 3 substitutents each selected independently from nitro, cyano, halo, trifluoromethyl, carbo(lower)alkoxy, acetyl, or carbamoyl, unsubstituted or substituted with lower alkyl, acetoxy, carboxy, hydroxy, amino, lower alkylamino, lower acylamino, or lower alkoxy; (ii) a vicinally divalent residue of pyridine, pyrrolidine, imidazole, naphthalene, or thiophene, wherein the divalent bonds are on vicinal ring carbon atoms; (iii) a vicinally divalent cycloalkyl or cycloalkenyl of 4-10 carbon atoms, unsubstituted or substituted with 1 or more substitutents each selected independently from the group consisting of nitro, cyano, halo, trifluoromethyl, carbo(lower)alkoxy, acetyl, carbamoyl, acetoxy, carboxy, hydroxy, amino, lower alkylamino, lower alkyl, lower alkoxy, or phenyl; (iv) vinylene di-substituted with lower alkyl; or (v) ethylene, unsubstituted or monosubstituted or disubstituted with lower alkyl;

R⁶ is —CO—, —CH₂—, or —CH₂CO—;

Y is —COX, —C≡N, —OR⁸, alkyl of 1 to 5 carbon atoms, or aryl;

X is —NH₂, —OH, —NHR, —R⁹, —OR⁹, or alkyl of 1 to 5 carbon atoms;

R⁸ is hydrogen or lower alkyl;

R⁹ is alkyl or benzyl; and,

n has a value of 0, 1, 2, or 3.

Other representative compounds are of formula:

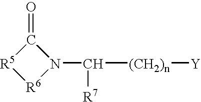

wherein:

Y is —C≡N or CO(CH₂)ₘCH₃;

m is 0, 1, 2, or 3;

R⁵ is (i) o-phenylene, unsubstituted or substituted with 1 to 3 substitutents each selected independently from nitro, cyano, trifluoromethyl, carbethoxy, carbomethoxy, carbopropoxy, acetyl, carbamoyl, carbamoyl substituted with and alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, or halo; (ii) the divalent residue of pyridine, pyrrolidine, imidizole, naphthalene, or thiophene, wherein the divalent bonds are on vicinal ring carbon atoms; (iii) a divalent cycloalkyl of 4-10 carbon atoms, unsubstituted or substituted with one or more substitutents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, phenyl or halo; (iv) di-substituted vinylene, substituted with nitro, cyano, trifluoromethyl, carbethoxy, carbomethoxy, carbopropoxy, acetyl, carbamoyl, carbamoyl substituted with and alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, or halo; or (v) ethylene, unsubstituted or substituted with 1 to 2 substitutents each selected independently from nitro, cyano, trifluoromethyl, carbethoxy, carbomethoxy, carbopropoxy, acetyl, carbamoyl, carbamoyl substituted with and alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, or halo;

R⁶ is —CO—, —CH₂—, —CH₂CO—, or —SO₂—;

R⁷ is (i) straight or branched alkyl of 1 to 12 carbon atoms; (ii) cyclic or bicyclic alkyl of 1 to 12 carbon atoms; (iii) pyridyl; (iv) phenyl substituted with one or more substitutents each selected independently of the other from nitro, cyano, trifluoromethyl, carbethoxy, carbomethoxy, carbopropoxy, acetyl, carbamoyl, acetoxy, carboxy, hydroxy, amino, straight, branched, cyclic, or bicyclic alkyl of 1 to 10 carbon atoms, straight, branched, cyclic, or bicyclic alkoxy of 1 to 10 carbon atoms, CH₂R where R is a cyclic or bicyclic alkyl of 1 to 10 carbon atoms, or halo; (v) benzyl substituted with one to three substitutents each selected independently from the group consisting of nitro, cyano, trifluoromethyl, carbethoxy, carbomethoxy, carbopropoxy, acetyl, carbamoyl, acetoxy, carboxy, hydroxy, amino, alkyl of 1 to 4 carbon atoms, alkoxy of 1 to 10 carbon atoms, or halo; (vi) naphthyl; or (vii) benzyloxy; and

n has a value of 0, 1, 2, or 3.

In another embodiment, specific selective cytokine inhibitory drugs are of formula:

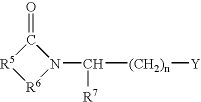

wherein:

R⁵ is (i) the divalent residue of pyridine, pyrrolidine, imidizole, naphthalene, or thiophene, wherein the divalent bonds are on vicinal ring carbon atoms; (ii) a divalent cycloalkyl of 4-10 carbon atoms, unsubstituted or substituted with one or more substitutents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, phenyl or halo; (iii) di-substituted vinylene, substituted with nitro, cyano, trifluoromethyl, carbethoxy, carbomethoxy, carbopropoxy, acetyl, carbamoyl, carbamoyl substituted with and alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, or halo; or (iv) ethylene, unsubstituted or substituted with 1 to 2 substitutents each selected independently from nitro, cyano, trifluoromethyl, carbethoxy, carbomethoxy, carbopropoxy, acetyl, carbamoyl, carbamoyl substituted with and alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, or halo;

R⁶ is —CO—, —CH₂—, —CH₂CO—, or —SO₂—;

R⁷ is (i) cyclic or bicyclic alkyl of 4 to 12 carbon atoms; (ii) pyridyl; (iii) phenyl substituted with one or more substitutents each selected independently of the other from nitro, cyano, trifluoromethyl, carbethoxy, carbomethoxy, carbopropoxy, acetyl, carbamoyl, acetoxy, carboxy, hydroxy, amino, straight, branched, cyclic, or bicyclic alkyl of 1 to 10 carbon atoms, straight, branched, cyclic, or bicyclic alkoxy of 1 to 10 carbon atoms, CH₂R where R is a cyclic or bicyclic alkyl of 1 to 10 carbon atoms, or halo; (iv) benzyl substituted with one to three substitutents each selected independently from the group consisting of nitro, cyano, trifluoromethyl, carbethoxy, carbomethoxy, carbopropoxy, acetyl, carbamoyl, acetoxy, carboxy, hydroxy, amino, alkyl of 1 to 4 carbon atoms, alkoxy of 1 to 10 carbon atoms, or halo; (v) naphthyl; or (vi) benzyloxy; and

Y is COX, —C≡N, OR⁸, alkyl of 1 to 5 carbon atoms, or aryl;

X is —NH₂, —OH, —NHR, —R⁹, —OR⁹, or alkyl of 1 to 5 carbon atoms;

R⁸ is hydrogen or lower alkyl;

R⁹ is alkyl or benzyl; and

n has a value of 0, 1, 2, or 3.

Other specific selective cytokine inhibitory drugs include, but are not limited to, the aryl amides (for example, an embodiment being N-benzoyl-3-amino-3-(3′,4′-dimethoxyphenyl)-propanamide) of U.S. Pat. Nos. 5,801,195, 5,736,570, 6,046,221 and 6,284,780, each of which is incorporated herein by reference. Representative compounds are of formula:

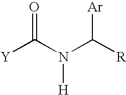

wherein:

Ar is (i) straight, branched, or cyclic, unsubstituted alkyl of 1 to 12 carbon atoms; (ii) straight, branched, or cyclic, substituted alkyl of 1 to 12 carbon atoms; (iii) phenyl; (iv) phenyl substituted with one or more substitutents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, or halo; (v) heterocycle; or (vi) heterocycle substituted with one or more substitutents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo;

R is —H, alkyl of 1 to 10 carbon atoms, CH₂OH, CH₂CH₂OH, or CH₂COZ where Z is alkoxy of 1 to 10 carbon atoms, benzyloxy, or NHR¹ where R¹ is H or alkyl of 1 to 10 carbon atoms; and

Y is i) a phenyl or heterocyclic ring, unsubstituted or substituted one or more substitutents each selected independently one from the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo or ii) naphthyl. Specific examples of the compounds are of formula:

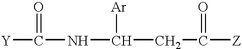

wherein:

Ar is 3,4-disubstituted phenyl where each substitutent is selected independently of the other from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo;

Z is alkoxy of 1 to 10 carbon atoms, benzyloxy, amino, or alkylamino of 1 to 10 carbon atoms; and

Y is (i) a phenyl, unsubstituted or substituted with one or more substitutents each selected, independently one from the other, from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo, or (ii) naphthyl.

Other specific selective cytokine inhibitory drugs include, but are not limited to, the imide/amide ethers and alcohols (for example, 3-phthalimido-3-(3′,4′-dimethoxyphenyl) propan-1-ol) disclosed in U.S. Pat. No. 5,703,098, which is incorporated herein by reference. Representative compounds have the formula:

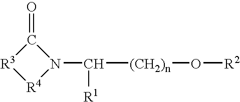

wherein:

R¹ is (i) straight, branched, or cyclic, unsubstituted alkyl of 1 to 12 carbon atoms; (ii) straight, branched, or cyclic, substituted alkyl of 1 to 12 carbon atoms; (iii) phenyl; or (iv) phenyl substituted with one or more substitutents each selected independently of the other from the group consisting of nitro, cyano, trifluoromethyl, carbethoxy, carbomethoxy, carbopropoxy, acetyl, carbamoyl, acetoxy, carboxy, hydroxy, amino, acylamino, alkylamino, di(alkyl)amino, alkyl of 1 to 10 carbon atoms, cycloalkyl of 3 to 10 carbon atoms, bicycloalkyl of 5 to 12 carbon atoms, alkoxy of 1 to 10 carbon atoms, cycloalkoxy of 3 to 10 carbon atoms, bicycloalkoxy of 5 to 12 carbon atoms, and halo;

R² is hydrogen, alkyl of 1 to 8 carbon atoms, benzyl, pyridylmethyl, or alkoxymethyl;

R³ is (i) ethylene, (ii) vinylene, (iii) a branched alkylene of 3 to 10 carbon atoms, (iv) a branched alkenylene of 3 to 10 carbon atoms, (v) cycloalkylene of 4 to 9 carbon atoms unsubstituted or substituted with one or more substitutents each selected independently from the group consisting of nitro, cyano, trifluoromethyl, carbethoxy, carbomethoxy, carbopropoxy, acetyl, carbamoyl, acetoxy, carboxy, hydroxy, amino, amino substituted with alkyl of 1 to 6 carbon atoms, amino substituted with acyl of 1 to 6 carbon atoms, alkyl of 1 to 10 carbon atoms, alkoxy of 1 to 12 carbon atoms, and halo, (vi) cycloalkenylene of 4 to 9 carbon atoms unsubstituted or substituted with one or more substitutents each selected independently from the group consisting of nitro, cyano, trifluoromethyl, carbethoxy, carbomethoxy, carbopropoxy, acetyl, carbamoyl, acetoxy, carboxy, hydroxy, amino, amino substituted with alkyl of 1 to 6 carbon atoms, amino substituted with acyl of 1 to 6 carbon atoms, alkyl of 1 to 10 carbon atoms, alkoxy of 1 to 12 carbon atoms, and halo, (vii) o-phenylene unsubstituted or substituted with one or more substitutents each selected independently from the group consisting of nitro, cyano, trifluoromethyl, carbethoxy, carbomethoxy, carbopropoxy, acetyl, carbamoyl, acetoxy, carboxy, hydroxy, amino, amino substituted with alkyl of 1 to 6 carbon atoms, amino substituted with acyl of 1 to 6 carbon atoms, alkyl of 1 to 10 carbon atoms, alkoxy of 1 to 12 carbon atoms, and halo, (viii) naphthyl, or (ix) pyridyl;

R⁴ is —CX—, —CH₂— or —CH₂CX—;

X is O or S; and

n is 0, 1, 2, or 3.

Other specific selective cytokine inhibitory drugs include, but are not limited to, the succinimides and maleimides (for example methyl 3-(3′,4′,5′6′-petrahydrophthalimdo)-3-(3″,4″-dimethoxyphenyl)propionate) disclosed in U.S. Pat. No. 5,658,940, which is incorporated herein by reference. Representative compounds are of formula:

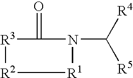

wherein:

R¹ is —CH₂—, —CH₂CO—, or —CO—;

R² and R³ taken together are (i) ethylene unsubstituted or substituted with alkyl of 1-carbon atoms or phenyl, (ii) vinylene substituted with two substitutents each selected, independently of the other, from the group consisting of alkyl of 1-10 carbon atoms and phenyl, or (iii) a divalent cycloalkyl of 5-10 carbon atoms, unsubstituted or substituted with one or more substitutents each selected independently of the other from the group consisting of nitro, cyano, trifluoromethyl, carbethoxy, carbomethoxy, carbopropoxy, acetyl, carbamoyl unsubstituted or substituted with alkyl of 1-3 carbon atoms, acetoxy, carboxy, hydroxy, amino, substituted amino, alkyl of 1 to 10 carbon atoms, alkoxy of 1 to 10 carbon atoms, norbornyl, phenyl or halo;

R⁴ is (i) straight or branched unsubstituted alkyl of 4 to 8 carbon atoms, (ii) cycloalkyl or bicycloalkyl of 5-10 carbon atoms, unsubstituted or substituted with one or more substitutents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, branched, straight or cyclic alkyl of 1 to 10 carbon atoms, alkoxy of 1 to 10 carbon atoms, phenyl or halo, (iii) phenyl substituted with one or more substitutents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, cycloalkyl or bicycloalkyl of 3 to 10 carbon atoms, cycloalkoxy or bicycloalkoxy of 3 to 10 carbon atoms, phenyl or halo, (iv) pyridine or pyrrolidine, unsubstituted or substituted with one or more substitutents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, phenyl or halo; and,

R⁵ is —COX, —CN, —CH₂COX, alkyl of 1 to 5 carbon atoms, aryl, —CH₂OR, —CH₂ aryl, or —CH₂OH,

where X is NH₂, OH, NHR, or OR⁶,

where R is lower alkyl; and

where R⁶ is alkyl or benzyl.

Other specific selective cytokine inhibitory drugs include, but are not limited to, substituted imides (for example, 2-phthalimido-3-(3′,4′-dimethoxyphenyl)propane) disclosed in U.S. Pat. No. 6,429,221, which is incorporated herein by reference. Representative compounds have the formula:

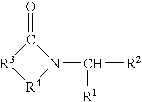

wherein:

R¹ is (i) straight, branched, or cyclic alkyl of 1 to 12 carbon atoms, (ii) phenyl or phenyl substituted with one or more substitutents each selected independently of the other from nitro, cyano, trifluoromethyl, carbethoxy, carbomethoxy, carbopropoxy, acetyl, carbamoyl, acetoxy, carboxy, hydroxy, amino, straight or branched alkyl of 1 to 10 carbon atoms, alkoxy of 1 to 10 carbon atoms, or halo, (iii) benzyl or benzyl substituted with one or more substitutents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo, or (iv) —Y-Ph where Y is a straight, branched, or cyclic alkyl of 1 to 12 carbon atoms and Ph is phenyl or phenyl substituted with one or more substitutents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo;

R² is —H, a branched or unbranched alkyl of 1 to 10 carbon atoms, phenyl, pyridyl, heterocycle, —CH₂-aryl, or —CH₂-heterocycle;

R³ is i) ethylene, ii) vinylene, iii) a branched alkylene of 3 to 10 carbon atoms, iv) a branched alkenylene of 3 to 10 carbon atoms, v) cycloalkylene of 4 to 9 carbon atoms unsubstituted or substituted with 1 to 2 substitutents each selected independently from nitro, cyano, trifluoromethyl, carbethoxy, carbomethoxy, carbopropoxy, acetyl, carbamoyl, acetoxy, carboxy, hydroxy, amino, substituted amino, alkyl of 1 to 4 carbon atoms, alkoxy of 1 to 4 carbon atoms, or halo, vi) cycloalkenylene of 4 to 9 carbon atoms unsubstituted or substituted with 1 to 2 substitutents each selected independently from nitro, cyano, trifluoromethyl, carbethoxy, carbomethoxy, carbopropoxy, acetyl, carbamoyl, acetoxy, carboxy, hydroxy, amino, substituted amino, alkyl of 1 to 4 carbon atoms, alkoxy of 1 to 4 carbon atoms, or halo, or vii) o-phenylene unsubstituted or substituted with 1 to 2 substitutents each selected independently from nitro, cyano, trifluoromethyl, carbethoxy, carbomethoxy, carbopropoxy, acetyl, carbamoyl, acetoxy, carboxy, hydroxy, amino, substituted amino, alkyl of 1 to 4 carbon atoms, alkoxy 1 to 4 carbon atoms, or halo; and,

R⁴ is —CX, or —CH₂—;

X is O or S.

Other specific selective cytokine inhibitory drugs include, but are not limited to, substituted 1,3,4-oxadiazoles (for example, 2-[1-(3-cyclopentyloxy-4-methoxyphenyl)-2-(1,3,4-oxadiazole-2-yl)ethyl]-5-methylisoindoline-1,3-dione) disclosed in U.S. Pat. No. 6,326,388, which is incorporated herein by reference. Representative compounds are of formula:

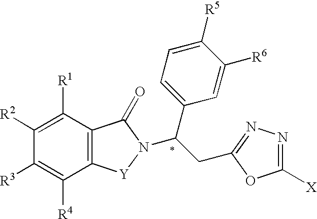

wherein:

the carbon atom designated constitutes a center of chirality;

Y is C═O, CH₂, SO₂ or CH₂C═O;

X is hydrogen, or alkyl of 1 to 4 carbon atoms;

each of R¹, R², R³, and R⁴, independently of the others, is hydrogen, halo, trifluoromethyl, acetyl, alkyl of 1 to 8 carbon atoms, alkoxy of 1 to 4 carbon atoms, nitro, cyano, hydroxy, —CH₂NR⁸R⁹, —(CH₂)₂NR⁸R⁹, or —NR⁸R⁹ or

any two of R¹, R², R³, and R⁴ on adjacent carbon atoms, together with the depicted benzene ring are naphthylidene, quinoline, quinoxaline, benzimidazole, benzodioxole or 2-hydroxybenzimidazole;

each of R⁵ and R⁶, independently of the other, is hydrogen, alkyl of 1 to 4 carbon atoms, alkoxy of 1 to 6 carbon atoms, cyano, benzocycloalkoxy, cycloalkoxy of up to 18 carbon atoms, bicyloalkoxy of up to 18 carbon atoms, tricylcoalkoxy of up to 18 carbon atoms, or cycloalkylalkoxy of up to 18 carbon atoms;

each of R⁸ and R⁹, taken independently of the other is hydrogen, straight or branched alkyl of 1 to 8 carbon atoms, phenyl, benzyl, pyridyl, pyridylmethyl, or one of R⁸ and R⁹ is hydrogen and the other is —COR¹⁰, or —SO₂R¹⁰, or R⁸ and R⁹ taken together are tetramethylene, pentamethylene, hexamethylene, —CH═NCH═CH—, or —CH₂CH₂X¹CH₂CH₂— in which X¹ is —O—, —S—, or —NH—,

R¹⁰ is hydrogen, alkyl of 1 to 8 carbon atoms, cycloalkyl, cycloalkylmethyl of up to 6 carbon atoms, phenyl, pyridyl, benzyl, imidazolylmethyl, pyridylmethyl, NR¹¹R¹², CH₂R¹⁴R¹⁵, or NR¹¹R¹²

wherein R¹⁴ and R¹⁵, independently of each other, are hydrogen, methyl, ethyl, or propyl, and

wherein R¹¹ and R¹², independently of each other, are hydrogen, alkyl of 1 to 8 carbon atoms, phenyl, or benzyl; and

the acid addition salts of said compounds which contain a nitrogen atom susceptible of protonation.

Specific examples of the compounds are of formula:

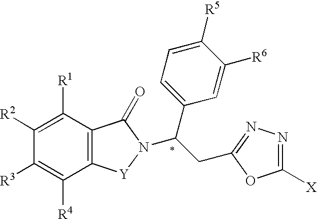

wherein:

the carbon atom designated constitutes a center of chirality;

Y is C═O, CH₂, SO₂ or CH₂C═O;

X is hydrogen, or alkyl of 1 to 4 carbon atoms;

(i) each of R¹, R², R³, and R⁴, independently of the others, is hydrogen, halo, trifluoromethyl, acetyl, alkyl of 1 to 8 carbon atoms, alkoxy of 1 to 4 carbon atoms, nitro, cyano, hydroxy, —CH₂NR⁸R⁹, —(CH₂)₂NR⁸R⁹, or —NR⁸R⁹ or

(ii) any two of R¹, R², R³, and R⁴ on adjacent carbon atoms, together with the depicted benzene ring to which they are bound are naphthylidene, quinoline, quinoxaline, benzimidazole, benzodioxole or 2-hydroxybenzimidazole;

each of R⁵ and R⁶, independently of the other, is hydrogen, alkyl of 1 to 4 carbon atoms, alkoxy of 1 to 6 carbon atoms, cyano, benzocycloalkoxy, cycloalkoxy of up to 18 carbon atoms, bicyloalkoxy of up to 18 carbon atoms, tricylcoalkoxy of up to 18 carbon atoms, or cycloalkylalkoxy of up to 18 carbon atoms;

(i) each of R⁸ and R⁹, independently of the other, is hydrogen, alkyl of 1 to 8 carbon atoms, phenyl, benzyl, pyridyl, pyridylmethyl, or

(ii) one of R⁸ and R⁹ is hydrogen and the other is —COR¹⁰, or —SO₂R¹⁰, in which R¹⁰ is hydrogen, alkyl of 1 to 8 carbon atoms, cycloalkyl, cycloalkylmethyl of up to 6 carbon atoms, phenyl, pyridyl, benzyl, imidazolylmethyl, pyridylmethyl, NR¹¹R¹², or CH₂NR¹⁴R¹⁵, wherein R¹¹ and R¹², independently of each other, are hydrogen, alkyl of 1 to 8 carbon atoms, phenyl, or benzyl and R¹⁴ and R¹⁵, independently of each other, are hydrogen, methyl, ethyl, or propyl; or

(iii) R⁸ and R⁹ taken together are tetramethylene, pentamethylene, hexamethylene, —CH═NCH═CH—, or —CH₂CH₂X¹CH₂CH₂— in which X¹ is —O—, —S—, or —NH—.

Other specific selective cytokine inhibitory drugs include, but are not limited to, cyano and carboxy derivatives of substituted styrenes (for example, 3,3-bis-(3,4-dimethoxyphenyl)acrylonitrile) disclosed in U.S. Pat. Nos. 5,929,117, 6,130,226, 5,262,101 and 6,479,554, each of which is incorporated herein by reference. Representative compounds are of formula:

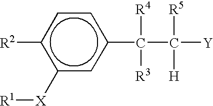

wherein:

(a) X is —O— or —(CₙH₂ₙ)— in which n has a value of 0, 1, 2, or 3, and R¹ is alkyl of one to 10 carbon atoms, monocycloalkyl of up to 10 carbon atoms, polycycloalkyl of up to 10 carbon atoms, or benzocyclic alkyl of up to 10 carbon atoms, or

(b) X is —CH═ and R¹ is alkylidene of up to 10 carbon atoms, monocycloalkylidene of up to 10 carbon atoms, or bicycloalkylidene of up to 10 carbon atoms;

R² is hydrogen, nitro, cyano, trifluoromethyl, carbethoxy, carbomethoxy, carbopropoxy, acetyl, carbamoyl, acetoxy, carboxy, hydroxy, amino, lower alkyl, lower alkylidenemethyl, lower alkoxy, or halo;

R³ is (i) phenyl, unsubstituted or substituted with 1 or more substitutents each selected independently from nitro, cyano, halo, trifluoromethyl, carbethoxy, carbomethoxy, carbopropoxy, acetyl, carbamoyl, carbamoyl substituted with alkyl of 1 to 3 carbon atoms, acetoxy, carboxy, hydroxy, amino, amino substituted with an alkyl of 1 to 5 carbon atoms, alkyl of up to 10 carbon atoms, cycloalkyl of up to 10 carbon atoms, alkoxy of up to 10 carbon atoms, cycloalkoxy of up to 10 carbon atoms, alkylidenemethyl of up to 10 carbon atoms, cycloalkylidenemethyl of up to 10 carbon atoms, phenyl, or methylenedioxy; (ii) pyridine, substituted pyridine, pyrrolidine, imidizole, naphthalene, or thiophene; (iii) cycloalkyl of 4-10 carbon atoms, unsubstituted or substituted with 1 or more substitutents each selected independently from the group consisting of nitro, cyano, halo, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, phenyl;

each of R⁴ and R⁵ taken individually is hydrogen or R⁴ and R⁵ taken together are a carbon-carbon bond;

Y is —COZ, —C≡N, or lower alkyl of 1 to 5 carbon atoms;

Z is —OH, —NR⁶R⁶, —R⁷, or —OR⁷; R⁶ is hydrogen or lower alkyl; and R⁷ is alkyl or benzyl. Specific examples of the compounds are of formula:

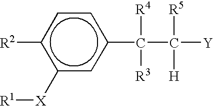

wherein:

(a) X is —O— or —(CₙH₂ₙ)— in which n has a value of 0, 1, 2, or 3, and R¹ is alkyl of one to 10 carbon atoms, monocycloalkyl of up to 10 carbon atoms, polycycloalkyl of up to 10 carbon atoms, or benzocyclic alkyl of up to 10 carbon atoms, or

(b) X is —CH═ and R¹ is alkylidene of up to 10 carbon atoms, monocycloalkylidene of up to 10 carbon atoms, or bicycloalkylidene of up to 10 carbon atoms;

R² is hydrogen, nitro, cyano, trifluoromethyl, carbethoxy, carbomethoxy, carbopropoxy, acetyl, carbamoyl, acetoxy, carboxy, hydroxy, amino, lower alkyl, lower alkylidenemethyl, lower alkoxy, or halo;

R³ is pyrrolidine, imidazole or thiophene unsubstituted or substituted with 1 or more substitutents each selected independently from the group consisting of nitro, cyano, halo, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, or phenyl;

each of R⁴ and R⁵ taken individually is hydrogen or R⁴ and R⁵ taken together are a carbon-carbon bond;

Y is —COZ, —C≡N, or lower alkyl of 1 to 5 carbon atoms;

Z is —OH, —NR⁶R⁶, —R⁷, or —OR⁷; R⁶ is hydrogen or lower alkyl; and R⁷ is alkyl or benzyl.

Particularly preferred nitriles are compounds of the formula:

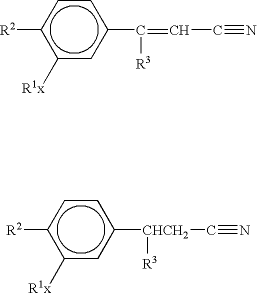

wherein:

(a) X is —O— or —(CₙH₂ₙ)— in which n has a value of 0, 1, 2, or 3, and R¹ is alkyl of up to 10 carbon atoms, monocycloalkyl of up to 10 carbon atoms, polycycloalkyl of up to 10 carbon atoms, or benzocyclic alkyl of up to 10 carbon atoms, or

(b) X is —CH═, and R¹ is alkylidene of up to 10 carbon atoms or monocycloalkylidene of up to 10 carbon atoms;

R² is hydrogen, nitro, cyano, trifluoromethyl, carbethoxy, carbomethoxy, carbopropoxy, acetyl, carbamoyl, acetoxy, carboxy, hydroxy, amino, lower alkyl, lower alkoxy, or halo; and

R³ is (i) phenyl or naphthyl, unsubstituted or substituted with 1 or more substitutents each selected independently from nitro, cyano, halo, trifluoromethyl, carbethoxy, carbomethoxy, carbopropoxy, acetyl, carbamoyl, or carbamoyl substituted with alkyl of 1 to 3 carbon atoms, acetoxy, carboxy, hydroxy, amino, amino substituted with an alkyl of 1 to 5 carbon atoms, alkoxy or cycloalkoxy of 1 to 10 carbon atoms; or (ii) cycloalkyl of 4 to 10 carbon atoms, unsubstituted or substituted with one or more substitutents each selected independently from the group consisting of nitro, cyano, halo, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, or phenyl.

Particularly preferred nitrile is of formula:

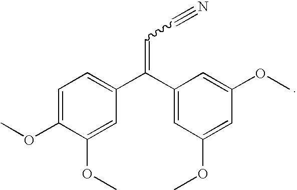

Other specific selective cytokine inhibitory drugs include, but are not limited to, isoindoline-1-one and isoindoline-1,3-dione substituted in the 2-position with an α-(3,4-disubstituted phenyl)alkyl group and in the 4- and/or 5-position with a nitrogen-containing group disclosed in WO 01/34606 and U.S. Pat. No. 6,667,316, which are incorporated herein by reference. Representative compounds are of formula:

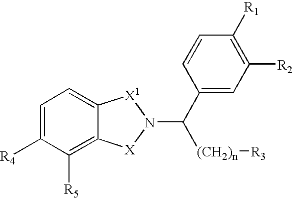

and include pharmaceutically acceptable salts and stereoisomers thereof,

wherein:

one of X and X′ is ═C═O or ═SO₂, and the other of X and X′ is ═C═O, ═CH₂, ═SO₂ or ═CH₂C═O;

n is 1, 2 or 3;

R₁ and R₂ are each independently (C₁-C₄)alkyl, (C₁-C₄)alkoxy, cyano, (C₃-C₁₈)cycloalkyl, (C₃-C₁₈)cycloalkoxy or (C₃-C₁₈)cycloalkyl-methoxy;

R₃ is SO₂—Y, COZ, CN or (C₁-C₆)hydroxyalkyl, wherein:

Y is (C₁-C₆)alkyl, benzyl or phenyl;

Z is —NR₆R₇, (C₁-C₆)alkyl, benzyl or phenyl;

R₆ is H, (C₁-C₄)alkyl, (C₃-C₁₈)cycloalkyl, (C₂-C₅)alkanoyl, benzyl or phenyl, each of which can be optionally substituted with halo, amino or (C₁-C₄)alkyl-amino;

R₇ is H or (C₁-C₄)alkyl;

R₄ and R₅ are taken together to provide —NH—CH₂—R₈—, NH—CO—R₈—, or —N═CH—R₈—, wherein:

R₈ is CH₂, O, NH, CH═CH, CH═N, or N═CH; or

one of R₄ and R₅ is H, and the other of R₄ and R₅ is imidazoyl, pyrrolyl, oxadiazolyl, triazolyl, or a structure of formula (A),

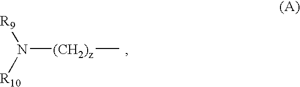

wherein:

z is 0 or 1;

R₉ is: H; (C₁-C₄)alkyl, (C₃-C₁₈)cycloalkyl, (C₂-C₅)alkanoyl, or (C₄-C₆)cycloalkanoyl, optionally substituted with halo, amino, (C₁-C₄)alkyl-amino, or (C₁-C₄)dialkyl-amino; phenyl; benzyl; benzoyl; (C₂-C₅)alkoxycarbonyl; (C₃-C₅)alkoxyalkylcarbonyl; N-morpholinocarbonyl; carbamoyl; N-substituted carbamoyl substituted with (C₁-C₄)alkyl; or methylsulfonyl; and

R¹⁰ is H, (C₁-C₄)alkyl, methylsulfonyl, or (C₃-C₅)alkoxyalkylcarbonyl; or

R⁹ and R¹⁰ are taken together to provide —CH═CH—CH═CH—, —CH═CH—N═CH—, or (C₁-C₂)alkylidene, optionally substituted with amino, (C₁-C₄)alkyl-amino, or (C₁-C₄)dialkyl-amino; or

R₄ and R₅ are both structures of formula (A).

In one embodiment, z is not 0 when (i) R³ is —SO₂—Y, —COZ, or —CN and (ii) one of R⁴ or R⁵ is hydrogen. In another embodiment, R⁹ and R¹⁰, taken together, is —CH═CH—CH═CH—, —CH═CH—N═CH—, or (C₁-C₂)alkylidene substituted by amino, (C₁-C₄)alkyl-amino, or (C₁-C₄)dialkyl-amino. In another embodiment, R₄ and R₅ are both structures of formula (A).

Specific compounds are of formula:

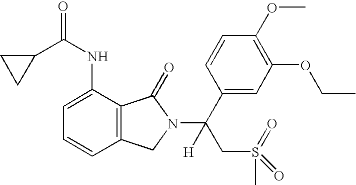

and the enantiomers thereof. Further specific compounds are of formulas:

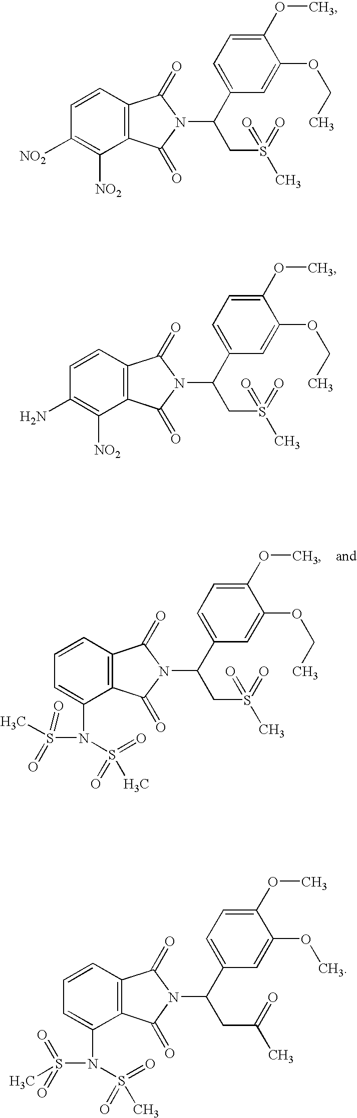

Further examples include, but are not limited to: 2-[1-(3-Ethoxy-4-methoxyphenyl)-2-methylsulfonylethyl]-4,5-dinitroisoindoline-1,3-dione; 2-[1-(3-Ethoxy-4-methoxyphenyl)-2-methylsulfonylethyl]-4,5-diaminoisoindoline-1,3-dione; 7-[1-(3-Ethoxy-4-methoxyphenyl)-2-methylsulfonylethyl]-3-pyrrolino[3,4-e]benzimidazole-6,8-dione; 7-[1-(3-Ethoxy-4-methoxyphenyl)-2-methylsulfonylethyl]hydro-3-pyrrolino[3,4-e]benzimidazole-2,6,8-trione; 2-[1-(3-Ethoxy-4-methoxyphenyl)-2-methylsulfonylethyl]-3-pyrrolino[3,4-f]quinoxaline-1,3-dione; Cyclopropyl-N-{2-[1-(3-ethoxy-4-methoxyphenyl)-2-methylsulfonylethyl]-1,3-dioxoisoindolin-4-yl}carboxamide; 2-Chloro-N-{2-[1-(3-ethoxy-4-methoxyphenyl)-2-methylsulfonylethyl]-1,3-dioxoisoindolin-4-yl}acetamide; 2-Amino-N-{2-[1-(3-ethoxy-4-methoxyphenyl)-2-methylsulfonylethyl]-1,3-dioxoisoindolin-4-yl}acetamide; 2-N,N-Dimethylamino-N-{2-[-(3-ethoxy-4-methoxyphenyl)-2-methylsulfonylethyl]-1,3-dioxoisoindolin-4-yl}acetamide; N-{2-[1-(3-ethoxy-4-methoxyphenyl)-2-methylsulfonylethyl]-1,3-dioxoisoindolin-4-yl}-2,2,2-trifluoroacetamide; N-{2-[1-(3-Ethoxy-4-methoxyphenyl)-2-methylsulfonylethyl]-1,3-dioxoisoindolin-4-yl}methoxycarboxamide; 4-[1-Aza-2-(dimethylamino)vinyl]-2-[1-(3-ethoxy-4-methoxyphenyl)-2-methylsulfonylethyl]isoindoline-1,3-dione; 4-[1-Aza-2-(dimethylamino)prop-1-enyl]-2-[1-(3-ethoxy-4-methoxyphenyl)-2-methylsulfonylethyl]isoindoline-1,3-dione; 2-[1-(3-Ethoxy-4-methoxyphenyl)-2-methylsulfonylethyl]-4-(5-methyl-1,3,4-oxadiazol-2-yl)isoindoline-1,3-dione; 2-[1-(3-Ethoxy-4-methoxyphenyl)-2-methylsulfonylethyl]-4-pyrrolylisoindoline-1,3-dione; 4-(Aminomethyl)-2-[-(3-ethoxy-4-methoxyphenyl)-2-methylsulfonylethyl]-isoindoline-1,3-dione; 2-[1-(3-Ethoxy-4-methoxyphenyl)-2-methylsulfonylethyl]-4-(pyrrolylmethyl)isoindoline-1,3-dione; N-{2-[1-(3-ethoxy-4-methoxyphenyl)-3-hydroxybutyl]-1,3-dioxoisoindolin-4-yl}acetamide; N-{2-[1-(3-Ethoxy-4-methoxyphenyl)-3-oxobutyl]-1,3-dioxoisoindolin-4-yl}acetamide; N-{2-[1R-(3-ethoxy-4-methoxyphenyl)-3-hydroxybutyl]-1,3-dioxoisoindolin-4-yl}acetamide; N-{2-[1R-(3-ethoxy-4-methoxyphenyl)-3-oxobutyl]-1,3-dioxoisoindolin-4-yl}acetamide; N-{2-[1S-(3-Ethoxy-4-methoxyphenyl)-3-hydroxybutyl]-1,3-dioxoisoindolin-4-yl}acetamide; N-{2-[1S-(3-ethoxy-4-methoxyphenyl)-3-oxobutyl]-1,3-dioxoisoindolin-4-yl}acetamide; 4-Amino-2-[1-(3-ethoxy-4-methoxyphenyl)-3-hydroxybutylisoindoline-1,3-dione; 4-Amino-2-[1-(3-ethoxy-4-methoxyphenyl)-3-oxobutyl]isoindoline-1,3-dione; 2-[1-(3-Ethoxy-4-methoxyphenyl)-3-oxobutyl]-4-pyrrolylisoindoline-1,3-dione; 2-Chloro-N-{2-[1-(3-ethoxy-4-methoxyphenyl)-3-oxobutyl]-1,3-dioxoisoindol-4-yl}acetamide; 2-(Dimethylamino)-N-{2-[1-(3-ethoxy-4-methoxyphenyl)-3-oxobutyl]-1,3-dioxoisoindolin-4-yl}acetamide; 4-Amino-2-[1R-(3-ethoxy-4-methoxyphenyl)-3-hydroxybutyl]isoindoline-1,3-dione; 4-Amino-2-[1R-(3-ethoxy-4-methoxyphenyl)-3-oxobutyl]isoindoline-1,3-dione; 2-[1R-(3-ethoxy-4-methoxyphenyl)-3-oxobutyl]-4-pyrrolylisoindoline-1,3-dione; 2-(Dimethylamino)-N-{2-[1R-(3-ethoxy-4-methoxyphenyl)-3-oxobutyl]-1,3-dioxoisoindolin-4-yl}acetamide; Cyclopentyl-N-{2-[1-(3-ethoxy-4-methoxyphenyl)-2-(methylsulfonyl)ethyl]-1,3-dioxoisoindolin-4-yl}carboxamide; 3-(Dimethylamino)-N-{2-[1-(3-ethoxy-4-methoxyphenyl)-2-(methylsulfonyl)ethyl]-1,3-dioxoisoindolin-4-yl}propanamide; 2-(Dimethylamino)-N-{2-[1-(3-ethoxy-4-methoxyphenyl)-2-(methylsulfonyl)ethyl]-1,3-dioxoisoindolin-4-yl}propanamide; N-{2-[(1R)-1-(3-ethoxy-4-methoxyphenyl)-2-(methylsulfonyl)ethyl]-1,3-dioxoisoindolin-4-yl}-2-(dimethylamino)acetamide; N-{2-[(1S)-1-(3-ethoxy-4-methoxyphenyl)-2-(methylsulfonyl)ethyl]-1,3-dioxoisoindolin-4-yl}-2-(dimethylamino)acetamide; 4-{3-[(Dimethylamino)methyl]pyrrolyl}-2-[1-(3-ethoxy-4-methoxyphenyl)-2-(methylsulfonyl)ethyl]isoindoline-1,3-dione; Cyclopropyl-N-{2-[(1S)-1-(3-ethoxy-4-methoxyphenyl)-2-(methylsulfonyl)ethyl]-1,3-dioxoisoindolin-4-yl}carboxamide; 2-[1-(3,4-dimethoxyphenyl)-2-(methylsulfonyl)ethyl]-4-pyrrolylisoindoline-1,3-dione; N-{2-[1-(3,4-dimethoxyphenyl)-2-(methylsulfonyl)ethyl]-1,3-dioxoisoindolin-4-yl}-2-(dimethylamino)acetamide; Cyclopropyl-N-{2-[1-(3,4-dimethoxyphenyl)-2-(methylsulfonyl)ethyl]-1,3-dioxoisoindolin-4-yl}carboxamide; Cyclopropyl-N-{2-[1-(3-ethoxy-4-methoxyphenyl)-2-(methylsulfonyl)ethyl]-3-oxoisoindolin-4-yl}carboxamide; 2-(Dimethylamino)-N-{2-[1-(3-ethoxy-4-methoxyphenyl)-2-(methylsulfonyl)ethyl]-3-oxoisoindolin-4-yl}acetamide; Cyclopropyl-N-{2-[(1S)-1-(3-ethoxy-4-methoxyphenyl)-2-(methylsulfonyl)ethyl]-3-oxoisoindolin-4-yl}carboxamide; Cyclopropyl-N-{2-[(1R)-1-(3-ethoxy-4-methoxyphenyl)-2-(methylsulfonyl)ethyl]-3-oxoisoindolin-4-yl}carboxamide; (3R)-3-[7-(Acetylamino)-1-oxoisoindolin-2-yl]-3-(3-ethoxy-4-methoxyphenyl)-N,N-dimethylpropanamide; (3R)-3-[7-(Cyclopropylcarbonylamino)-1-oxoisoindolin-2-yl]-3-(3-ethoxy-4-methoxyphenyl)-N,N-dimethylpropanamide; 3-{4-[2-(Dimethylamino)acetylamino]-1,3-dioxoisoindolin-2-yl}-3-(3-ethoxy-4-methoxyphenyl)-N,N-dimethylpropanamide; (3R)-3-[7-(2-Chloroacetylamino)-1-oxoisoindolin-2-yl]-3-(3-ethoxy-4-methoxy-phenyl)-N,N-dimethylpropanamide; (3R)-3-{4-[2-(dimethylamino)acetylamino]-1,3-dioxoisoindolin-2-yl}-3-(3-ethoxy-4-methoxyphenyl)-N,N-dimethylpropanamide; 3-(1,3-Dioxo-4-pyrrolylisoindolin-2-yl)-3-(3-ethoxy-4-methoxyphenyl)-N,N-dimethylpropanamide; 2-[1-(3-Ethoxy-4-methoxyphenyl)-2-(methylsulfonyl)ethyl]-4-(imidazolyl-methyl)isoindoline-1,3-dione; N-({2-[1-(3-Ethoxy-4-methoxyphenyl)-2-(methylsulfonyl)ethyl]-1,3-dioxoisoindolin-4-yl}methyl)acetamide; 2-Chloro-N-({2-[1-(3-ethoxy-4-methoxyphenyl)-2-(methylsulfonyl)ethyl]-1,3-dioxoisoindolin-4-yl}methyl)acetamide; 2-(Dimethylamino)-N-({2-[1-(3-ethoxy-4-methoxyphenyl)-2-(methylsulfonyl)ethyl]-1,3-dioxoisoindolin-4-yl}methyl)acetamide; 4-[Bis(methylsulfonyl)amino]-2-[1-(3-ethoxy-4-methoxyphenyl)-2-(methylsulfonyl)ethyl]isoindoline-1,3-dione; 2-[1-(3-Ethoxy-4-methoxyphenyl)-2-(methylsulfonyl)ethyl]-4-[(methylsulfonyl)amino]isoindoline-1,3-dione; N-{2-[1-(3-Ethoxy-4-methoxyphenyl)-3-hydroxypentyl]-1,3-dioxoisoindolin-4-yl}acetamide; N-{2-[1-(3-Ethoxy-4-methoxyphenyl)-3-oxopentyl]1,3-dioxoisoindolin-4-yl}acetamide; 2-[(1R)-1-(3-Ethoxy-4-methoxyphenyl)-3-hydroxybutyl]-4-(pyrrolylmethyl)isoindoline-1,3-dione; 2-[(1R)-1-(3-Ethoxy-4-methoxyphenyl)-3-oxobutyl]-4-(pyrrolylmethyl)isoindoline-1,3-dione; N-{2-[1-(3-Cyclopentyloxy-4-methoxyphenyl)-3-hydroxybutyl]-1,3-dioxoisoindolin-4-yl}acetamide; N-{2-[1-(3-Cyclopentyloxy-4-methoxyphenyl)-3-oxobutyl]-1,3-dioxoisoindolin-4-yl}acetamide; 2-[1-(3-Cyclopentyloxy-4-methoxyphenyl)-3-oxobutyl]-4-pyrrolylisoindoline-1,3-dione; 2-[1-(3,4-Dimethoxyphenyl)-3-oxobutyl]-4-[bis(methylsulfonyl)amino]isoindoline-1,3-dione; and pharmaceutically acceptable salts, solvates, and stereoisomers thereof.

Still other specific selective cytokine inhibitory drugs include, but are not limited to, imido and amido substituted acylhydroxamic acids (for example, (3-(1,3-dioxoisoindoline-2-yl)-3-(3-ethoxy-4-methoxyphenyl)propanoylamino)propanoate disclosed in WO 01/45702 and U.S. Pat. No. 6,699,899, which are incorporated herein by reference. Representative compounds are of formula:

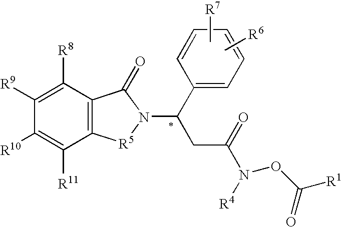

wherein:

the carbon atom designated * constitutes a center of chirality,

R⁴ is hydrogen or —(C═O)—R¹²,

each of R¹ and R², independently of each other, is alkyl of 1 to 6 carbon atoms, phenyl, benzyl, pyridyl methyl, pyridyl, imidazoyl, imidazolyl methyl, or

CHR*(CH₂)ₙNR*R⁰,

wherein R*and R⁰, independently of the other, are hydrogen, alkyl of 1 to 6 carbon atoms, phenyl, benzyl, pyridyl methyl, pyridyl, imidazoyl or imidazolylmethyl, and n=0, 1, or 2;

R⁵ is C═O, CH₂, CH₂—CO—, or SO₂;

each of R⁶ and R⁷, independently of the other, is nitro, cyano, trifluoromethyl, carbethoxy, carbomethoxy, carbopropoxy, acetyl, carbamoyl, acetoxy, carboxy, hydroxy, amino, alkyl of 1 to 6 carbon atoms, alkoxy of 1 to 6 carbon atoms, cycloalkoxy of 3 to 8 carbon atoms, halo, bicycloalkyl of up to 18 carbon atoms, tricycloalkoxy of up to 18 carbon atoms, 1-indanyloxy, 2-indanyloxy, C₄-C₈-cycloalkylidenemethyl, or C₃-C₁₀-alkylidenemethyl;

each of R⁸, R⁹, R¹⁰, and R¹¹, independently of the others, is (i) hydrogen, nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkoxy of 1 to 10 carbon atoms, halo, or

(ii) one of R⁸, R⁹, R¹⁰, and R¹¹ is acylamino comprising a lower alkyl, and the remaining of R⁸, R⁹, R¹⁰, and R¹¹ are hydrogen, or

(iii) hydrogen if R⁸ and R⁹ taken together are benzo, quinoline, quinoxaline, benzimidazole, benzodioxole, 2-hydroxybenzimidazole, methylenedioxy, dialkoxy, or dialkyl, or

(iv) hydrogen if R¹⁰ and R¹¹, taken together are benzo, quinoline, quinoxaline, benzimidazole, benzodioxole, 2-hydroxybenzimidazole, methylenedioxy, dialkoxy, or dialkyl, or

(v) hydrogen if R⁹ and R¹⁰ taken together are benzo.

Still specific selective cytokine inhibitory drugs include, but are not limited to, 7-amido-isoindolyl compounds disclosed in U.S. patent application Ser. No. 10/798,317 filed on Mar. 12, 2004, which is incorporated herein by reference. Representative compounds are of formula:

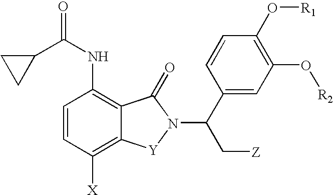

wherein:

Y is —C(O)—, —CH₂, —CH₂C(O)— or SO₂;

X is H;

Z is (C₀₋₄-alkyl)-C(O)R³, C₁₋₄-alkyl, (C₀₋₄-alkyl)-OH, (C₁₋₄-alkyl)-O(C₁₋₄-alkyl), (C₁₋₄-alkyl)-SO₂(C₁₋₄-alkyl), (C₀₋₄-alkyl)-SO(C₁₋₄-alkyl), (C₀₋₄-alkyl)-NH₂, (C₀₋₄-alkyl)-N(C₁₋₈alkyl)₂, (C₀₋₄-alkyl)-N(H)(OH), or CH₂NSO₂(C₁₋₄-alkyl);

R₁ and R₂ are independently C₁₋₈-alkyl, cycloalkyl, or (C₁₋₄-alkyl)cycloalkyl;

R³ is, NR⁴R⁵, OH, or O—(C₁₋₈-alkyl);

R⁴ is H;

R⁵ is —OH, or —OC(O)R⁶;

R⁶ is C₁₋₈-alkyl, amino-(C₁₋₈-alkyl), (C₁₋₈-alkyl)-(C₃₋₆-cycloalkyl), C₃₋₆-cycloalkyl, phenyl, benzyl, or aryl;

or a pharmaceutically acceptable salt, solvate, hydrate, stereoisomer, clathrate, or prodrug thereof; or formula:

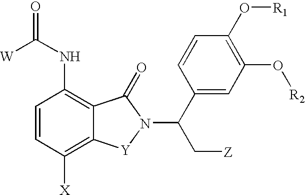

wherein:

Y is —C(O)—, —CH₂, —CH₂C(O)—, or SO₂;

X is halogen, —CN, —NR₇R₈, —NO₂, or —CF₃;

Z is (C₀₋₄alkyl)-SO₂(C₁₋₄-alkyl), —(C₀₋₄-alkyl)-CN, —(C₀₋₄-alkyl)-C(O)R³, C₁₋₄-alkyl, (C₀₋₄-alkyl)OH, (C₀₋₄-alkyl)O(C₁₋₄-alkyl), (C₀₋₄-alkyl)SO(C₁₋₄-alkyl), (C₀₋₄-alkyl)NH₂, (C₀₋₄-alkyl)N(C₁₋₈-alkyl)₂, (C₀₋₄-alkyl)N(H)(OH), (C₀₋₄-alkyl)-dichloropyridine or (C₀₋₄-alkyl)NSO₂(C₁₋₄-alkyl);

W is —C₃₋₆-cycloalkyl, —(C₁₋₈-alkyl)-(C₃₋₆-cycloalkyl), —(C₀₋₈-alkyl)-(C₃₋₆-cycloalkyl)-NR₇R₈, (C₀₋₈-alkyl)-NR₇R₈, (C₀₋₄alkyl)-CHR₉—(C₀₋₄alkyl)-NR₇R₈;

R₁ and R₂ are independently C₁₋₈-alkyl, cycloalkyl, or (C₁₋₄-alkyl)cycloalkyl;

R³ is C₁₋₈-alkyl, NR⁴R⁵, OH, or O—(C₁₋₈-alkyl);

R⁴ and R⁵ are independently H, C₁₋₈-alkyl, (C₀₋₈-alkyl)-(C₃₋₆-cycloalkyl), OH, or —OC(O)R⁶;

R⁶ is C₁₋₈-alkyl, (C₀₋₈-alkyl)-(C₃₋₆-cycloalkyl), amino-(C₁₋₈-alkyl), phenyl, benzyl, or aryl;

R₇ and R₈ are each independently H, C₁₋₈-alkyl, (C₀₋₈-alkyl)-(C₃₋₆-cycloalkyl), phenyl, benzyl, aryl, or can be taken together with the atom connecting them to form a 3 to 7 membered heterocycloalkyl or heteroaryl ring;

R₉ is C₁₋₄ alkyl, (C₀₋₄alkyl)aryl, (C₀₋₄alkyl)-(C₃₋₆-cycloalkyl), (C₀₋₄alkyl)-heterocylcle; or a pharmaceutically acceptable salt, solvate, hydrate, stereoisomer, clathrate, or prodrug thereof. In another embodiment, W is

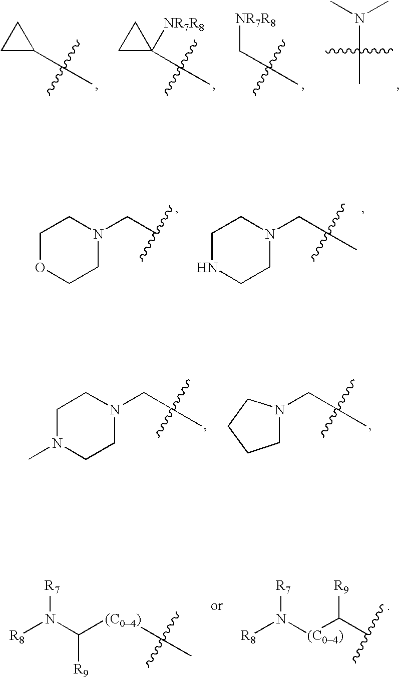

In another embodiment, representative compounds are of formula:

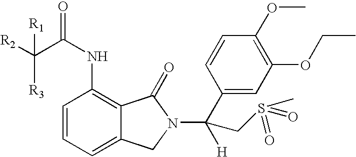

wherein:

R₁, R₂ and R₃ are independently H or C₁₋₈-alkyl, with the proviso that at least one of R₁, R₂ and R₃ is not H;

and pharmaceutically acceptable salts, solvates, hydrates, stereoisomers, clathrates, or prodrugs thereof.

Still specific selective cytokine inhibitory drugs include, but are not limited to, isoindoline compounds disclosed in U.S. patent application Ser. No. 10/900,332 filed on Jul. 28, 2004, which is incorporated herein by reference. Representative compounds are listed in Table 1 below, and pharmaceutically acceptable prodrugs, salts, solvates, and stereoisomers thereof:

TABLE 1

| No. | Structure |
|---|---|
| 1 | 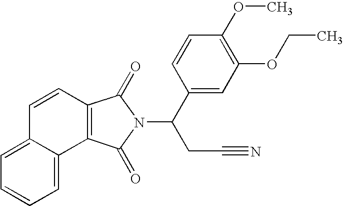 |
| 2 | 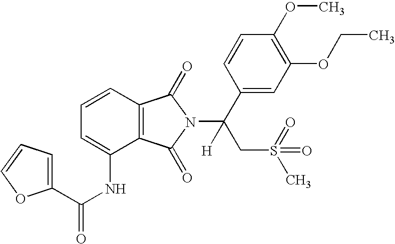 |
| 3 | 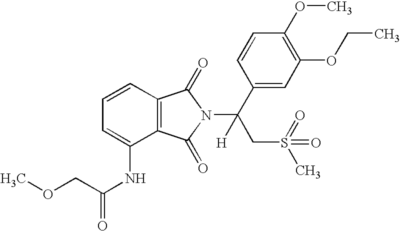 |
| 4 | 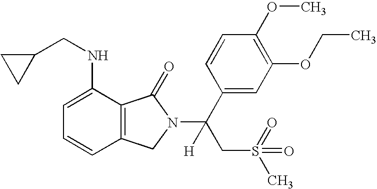 |
| 5 | 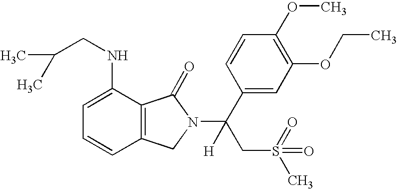 |
| 6 | 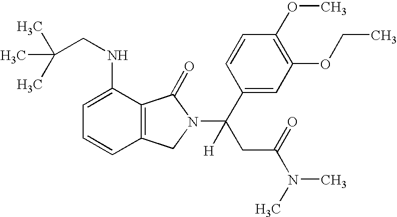 |
| 7 | 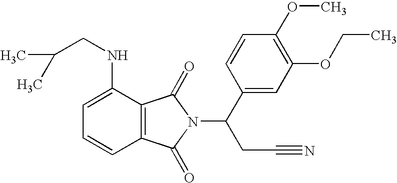 |
| 8 | 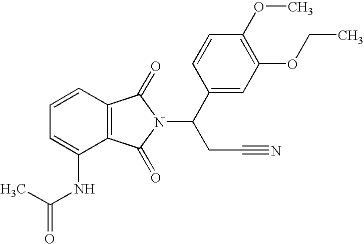 |
| 9 | 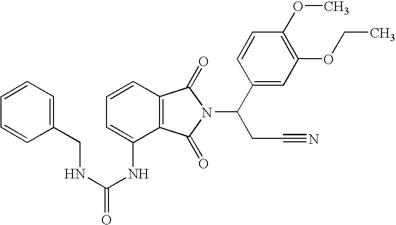 |
| 10 | 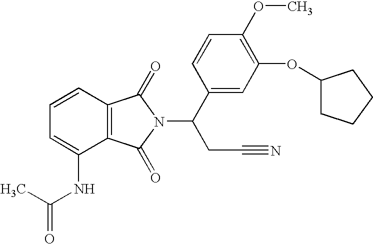 |
| 11 | 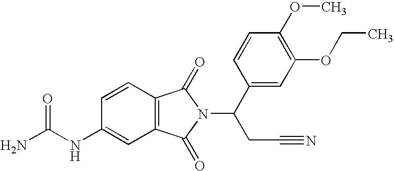 |
| 12 | 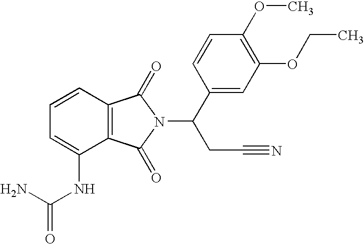 |
| 13 | 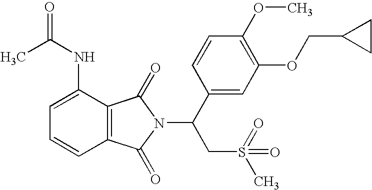 |
| 14 | 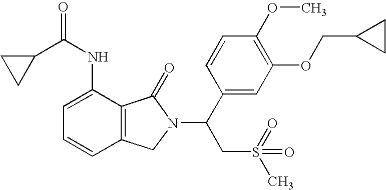 |
| 15 | 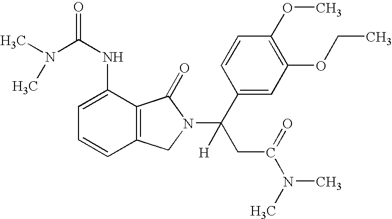 |
| 16 | 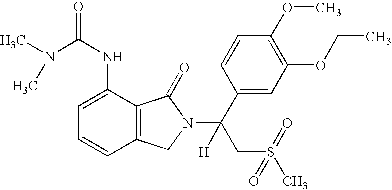 |

In another embodiment, this invention also encompasses 2-[1-(3-ethoxy-4-methoxyphenyl)-2-methylsulfonyethyl]-4,5-dinitroisoindoline-1,3-dione and its acid addition salts. In a particular embodiment, this invention encompasses a hydrochloride salt dione.

Still specific selective cytokine inhibitory drugs include, but are not limited to, isoindoline compounds disclosed in U.S. patent application Ser. No. 10/900,270 filed on Jul. 28, 2004, which is incorporated herein by reference. Representative compounds are cyclopropanecarboxylic acid {2-[1-(3-ethoxy-4-methoxy-phenyl)-2-[1,3,4]oxadiazol-2-yl-ethyl]-3-oxo-2,3-dihydro-1H-isoindol-4-yl}-amide, which has the following chemical structure, and pharmaceutically acceptable salts, solvates, prodrugs, and stereoisomers thereof:

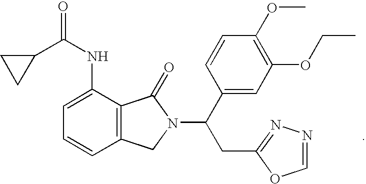

Still specific selective cytokine inhibitory drugs include, but are not limited to, N-alkyl-hydroxamic acid-isoindolyl compounds disclosed in U.S. provisional application No. 60/454,149 filed on Mar. 12, 2003, and its U.S. non-provisional application entitled “N-alkyl-hydroxamic acid-isoindolyl compounds and their pharmaceutical uses” which was filed on Mar. 12, 2004 by Man et al. under U.S. Ser. No. 10/798,372, each of which is incorporated herein by reference. Representative compounds are of formula:

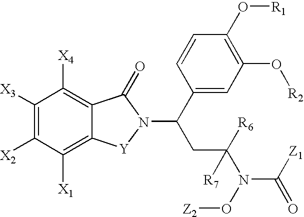

wherein:

Y is —C(O)—, —CH₂, —CH₂C(O)— or SO₂;

R₁ and R₂ are independently C₁₋₈-alkyl, CF₂H, CF₃, CH₂CHF₂, cycloalkyl, or (C₁₋₈-alkyl)cycloalkyl;

Z₁ is H, C₁₋₆-alkyl, —NH₂—NR₃R₄ or OR₅;

Z₂ is H or C(O)R₅;

X₁, X₂, X₃ and X₄ are each independent H, halogen, NO₂, OR₃, CF₃, C₁₋₆-alkyl, (C₀₋₄alkyl)-(C₃₋₆-cycloalkyl), (C₀₋₄-alkyl)-N—(R₈R₉), (C₀₋₄-alkyl)-NHC(O)—(R₈), (C₀₋₄-alkyl)-NHC(O)CH(R₈)(R₉), (C₀₋₄-alkyl)-NHC(O)N(R₈R₉), (C₀₋₄-alkyl)-NHC(O)O(R₈), (C₀₋₄-alkyl)-O—R₈, (C₀₋₄-alkyl)-imidazolyl, (C₀₋₄-alkyl)-pyrrolyl, (C₀₋₄-alkyl) oxadiazolyl, (C₀₋₄-alkyl)-triazolyl or (C₀₋₄-alkyl)-heterocycle;

R₃, R₄, and R₅ are each independently H, C₁₋₆-alkyl, O—C₁₋₆-alkyl, phenyl, benzyl, or aryl;

R₆ and R₇ are independently H or C₁₋₆-alkyl;

R₈ and R₉ are each independently H, C₁₋₉-alkyl, C₃₋₆-cycloalkyl, (C₁₋₆-alkyl)-(C₃₋₆-cycloalkyl), (C₀₋₆-alkyl)-N(R₄R₅), (C₁₋₆-alkyl)-OR₅, phenyl, benzyl, aryl, piperidinyl, piperizinyl, pyrrolidinyl, morpholino, or C₃₋₇-heterocycloalkyl; and

or a pharmaceutically acceptable salt, solvate, hydrate, stereoisomer, clathrate, or prodrug thereof.

Still specific selective cytokine inhibitory drugs include, but are not limited to, diphenylethylene compounds disclosed in U.S. patent application Ser. No. 10/794,931, filed on Mar. 5, 2004, which is incorporated herein by reference. Representative compounds are of formula:

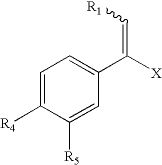

and pharmaceutically acceptable salts, solvates or hydrates thereof,

wherein:

R₁ is —CN, lower alkyl, —COOH, —C(O)—N(R₉)₂, —C(O)-lower alkyl, —C(O)-benzyl, —C(O)O-lower alkyl, —C(O)O-benzyl;

R₄ is —H, —NO₂, cyano, substituted or unsubstituted lower alkyl, substituted or unsubstituted alkoxy, halogen, —OH, —C(O)(R₁₀)₂, —COOH, —NH₂, —OC(O)—N(R₁₀)₂;

R₅ is substituted or unsubstituted lower alkyl, substituted or unsubstituted alkoxy, or substituted or unsubstituted alkenyl;

X is substituted or unsubstituted phenyl, substituted or unsubstituted pyridine, substituted or unsubstituted pyrrolidine, substituted or unsubstituted imidizole, substituted or unsubstituted naphthalene, substituted or unsubstituted thiophene, or substituted or unsubstituted cycloalkyl;

each occurrence of R₉ is independently —H or substituted or unsubstituted lower alkyl; and

each occurrence of R₁₀ is independently —H or substituted or unsubstituted lower alkyl. In another embodiment, representative compounds are of formula:

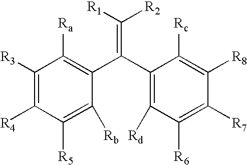

and pharmaceutically acceptable salts, solvates or hydrates thereof,

wherein:

R₁ and R₂ are independently —H, —CN, substituted or unsubstituted lower alkyl, substituted or unsubstituted alkenyl, substituted or unsubstituted alkynyl, —COOH, —C(O)-lower alkyl, —C(O)O-lower alkyl, —C(O)—N(R₉)₂, substituted or unsubstituted aryl, or substituted or unsubstituted heterocycle;

each occurrence of Rₐ, R_{b}, R_{c} and R_{d} is independently —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R₉)₂, —OC(O)—R₁₀, —OC(O)—R₁₀—N(R₁₀)₂, —C(O)N(R₁₀)₂, —NHC(O)—R₁₀, —NHS(O)₂—R₁₀, —S(O)₂—R₁₀, —NHC(O)NH—R₁₀, —NHC(O)N(R₁₀)₂, —NHC(O)NHSO₂—R₁₀, —NHC(O)—R₁₀—N(R₁₀)₂, —NHC(O)CH(R₁₀)(N(R₉)₂) or —NHC(O)—R₁₀—NH₂;

R₃ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R₉)₂, —OC(O)—R₁₀, —OC(O)—R₁₀—N(R₁₀)₂, —C(O)N(R₁₀)₂, —NHC(O)—R₁₀, —NHS(O)₂—R₁₀, —S(O)₂—R₁₀, —NHC(O)NH—R₁₀, —NHC(O)N(R₁₀)₂, —NHC(O)NHSO₂—R₁₀, —NHC(O)—R₁₀—N(R₁₀)₂, —NHC(O)CH(R₁₀)(N(R₉)₂) or —NHC(O)—R₁₀—NH₂, or R₃ with either Rₐ or with R₄, together form —O—C(R₁₆R₁₇)—O— or —O—(C(R₁₆R₁₇))₂—O—;

R₄ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R₉)₂, —OC(O)—R₁₀, —OC(O)—R₁₀—N(R₁₀)₂, —C(O)N(R₁₀)₂, —NHC(O)—R₁₀, —NHS(O)₂—R₁₀, —S(O)₂—R₁₀, —NHC(O)NH—R₁₀, —NHC(O)N(R₁₀)₂, —NHC(O)NHSO₂—R₁₀, —NHC(O)—R₁₀—N(R₁₀)₂, —NHC(O)CH(R₁₀)(N(R₉)₂) or —NHC(O)—R₁₀—NH₂;

R₅ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R₉)₂, —OC(O)—R₁₀, —OC(O)—R₁₀—N(R₁₀)₂, —C(O)N(R₁₀)₂, —NHC(O)—R₁₀, —NHS(O)₂—R₁₀, —S(O)₂—R₁₀, —NHC(O)NH—R₁₀, —NHC(O)N(R₁₀)₂, —NHC(O)NHSO₂—R₁₀, —NHC(O)—R₁₀—N(R₁₀)₂, —NHC(O)CH(R₁₀)(N(R₉)₂) or —NHC(O)—R₁₀—NH₂;

R₆ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R₉)₂, —OC(O)—R₁₀, —OC(O)—R₁₀—N(R₁₀)₂, —C(O)N(R₁₀)₂, —NHC(O)—R₁₀, —NHS(O)₂—R₁₀, —S(O)₂—R₁₀, —NHC(O)NH—R₁₀, —NHC(O)N(R₁₀)₂, —NHC(O)NHSO₂—R₁₀, —NHC(O)—R₁₀—N(R₁₀)₂, —NHC(O)CH(R₁₀)(N(R₉)₂) or —NHC(O)—R₁₀—NH₂;

R₇ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R₉)₂, —OC(O)—R₁₀, —OC(O)—R₁₀—N(R₁₀)₂, —C(O)N(R₁₀)₂, —NHC(O)—R₁₀, —NHS(O)₂—R₁₀, —S(O)₂—R₁₀, —NHC(O)NH—R₁₀, —NHC(O)N(R₁₀)₂, —NHC(O)NHSO₂—R₁₀, —NHC(O)—R₁₀—N(R₁₀)₂, —NHC(O)CH(R₁₀)(N(R₉)₂) or —NHC(O)—R₁₀—NH₂;

R₈ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R₉)₂, —OC(O)—R₁₀, —OC(O)—R₁₀—N(R₁₀)₂, —C(O)N(R₁₀)₂, —NHC(O)—R₁₀, —NHS(O)₂—R₁₀, —S(O)₂—R₁₀, —NHC(O)NH—R₁₀, —NHC(O)N(R₁₀)₂, —NHC(O)NHSO₂—R₁₀, —NHC(O)—R₁₀—N(R₁₀)₂, —NHC(O)CH(R₁₀)(N(R₉)₂) or —NHC(O)—R₁₀—NH₂, or R₈ with either R_{c} or with R₇, together form —O—C(R₁₆R₁₇)—O— or —O—(C(R₁₆R₁₇))₂—O—;

each occurrence of R₉ is independently —H, substituted or unsubstituted lower alkyl, or substituted or unsubstituted cycloalkyl;

each occurrence of R₁₀ is independently substituted or unsubstituted lower alkyl, substituted or unsubstituted cycloalkyl, substituted or unsubstituted aryl, substituted or unsubstituted lower hydroxyalkyl, or R₁₀ and a nitrogen to which it is attached form a substituted or unsubstituted heterocycle, or R₁₀ is —H where appropriate; and

each occurrence of R₁₆ and R₁₇ is independently —H or halogen.

Compounds of the invention can either be commercially purchased or prepared according to the methods described in the patents or patent publications disclosed herein. Further, optically pure compositions can be asymmetrically synthesized or resolved using known resolving agents or chiral columns as well as other standard synthetic organic chemistry techniques.

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases known in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of selective cytokine inhibitory drugs that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of a selective cytokine inhibitory drug that comprise —NO, —NO₂, —ONO, or —ONO₂ moieties. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, N.Y. 1985).

As used herein and unless otherwise indicated, the terms “biohydrolyzable amide,”“biohydrolyzable ester,” “biohydrolyzable carbamate,” “biohydrolyzable carbonate,”“biohydrolyzable ureide,” and “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkoxyacyloxyalkyl esters (such as methoxycarbonyloxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

Various selective cytokine inhibitory drugs contain one or more chiral centers, and can exist as racemic mixtures of enantiomers or mixtures of diastereomers. This invention encompasses the use of stereomerically pure forms of such compounds, as well as the use of mixtures of those forms. For example, mixtures comprising equal or unequal amounts of the enantiomers of selective cytokine inhibitory drugs may be used in methods and compositions of the invention. The purified (R) or (S) enantiomers of the specific compounds disclosed herein may be used substantially free of its other enantiomer.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

As used herein and unless otherwise indicated, the term “stereomerically enriched” means a composition that comprises greater than about 60% by weight of one stereoisomer of a compound, preferably greater than about 70% by weight, more preferably greater than about 80% by weight of one stereoisomer of a compound.

As used herein and unless otherwise indicated, the term “enantiomerically pure” means a stereomerically pure composition of a compound having one chiral center. Similarly, the term “enantiomerically enriched” means a stereomerically enriched composition of a compound having one chiral center.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

4.2 Second Active Ingredients

As discussed above, a second active ingredient or agent can be used in the methods and compositions of the invention together with selective cytokine inhibitory drugs, particularly conventional agents or therapies used to treat or manage central nervous system disorders. Specific second active agents also stimulate the division and differentiation of committed erythroid progenitors in cells in vitro or in vivo.

In one embodiment, a second active ingredient can be administered with a selective cytokine inhibitory drugs. In one embodiment, the second active ingredient is a dopamine agonist or antagonist, for example, but not limited to, Levodopa, L-DOPA/carbidopa combinations, cocaine, α-methyl-tyrosine, reserpine, tetrabenazine, benzotropine, pargyline, fenodolpam mesylate, cabergoline, pramipexole dihydrochloride, ropinorole, amantadine hydrochloride, selegiline hydrochloride, carbidopa, pergolide mesylate, Sinemet CR, or Symmetrel.

In another embodiment, the second active ingredient that is administered with a selective cytokine inhibitory drugs is a MAO, for example, but not limited to, iproniazid, clorgyline, phenelzine and isocarboxazid.

In another embodiment, the second active ingredient that is administered with a selective cytokine inhibitory drugs is a COMT, for example, but not limited to, tolcapone and entacapone.

In another embodiment, the second active ingredient that is administered with a selective cytokine inhibitory drugs is an acetylcholinesterase inhibitor, for example, but not limited to, tacrine, donepezil, rivastigmine, physostigmine saliclate, physostigmine sulfate, physostigmine bromide, meostigmine bromide, neostigmine methylsulfate, ambenonim chloride, edrophonium chloride, pralidoxime chloride, obidoxime chloride, trimedoxime bromide, diacetyl monoxim, endrophonium, pyridostigmine, and demecarium.

In yet another embodiment, the second active ingredient that is administered with a selective cytokine inhibitory drugs is an anti-inflammatory agent, including, but not limited to, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, Rₒ-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone and benzbromarone or betamethasone and other glucocorticoids.

In even another embodiment, the second active ingredient that is administered with a selective cytokine inhibitory drugs is an antiemetic agent, for example, but not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, and mixtures thereof.

4.3 Methods of Treatment and Management

Methods of this invention encompass methods of preventing, treating and/or managing central nervous system disorders. As used herein, unless otherwise specified, the term “preventing” includes but is not limited to, inhibition or the averting of symptoms associated with central nervous system disorders. Central nervous system disorders, include, but are not limited to, Parkinson disease; Alzheimer disease, mild cognitive impairment; depression; defective long-term memory; Amyotrophic Lateral Sclerosis (ALS); CNS trauma; hypokinetic disorders; bradykinesia; slowness of movement; paucity of movement; impairment of dexterity; hypophonia; monotonic speech; muscular rigidity; masked faces; decreased blinking; stooped posture; decreased arm swinging when walking; micrographia; parkinsonian tremor; parkinsonian gait; postural instability; festinating gait; motion freezing; disturbances of cognition, mood, sensation, sleep or autonomic function; dementia; and sleep disorders. As used herein, unless otherwise specified, the term “treating” refers to the administration of a composition after the onset of symptoms of central nervous system disorders, or a related disorder whereas “preventing” refers to the administration prior to the onset of symptoms, particularly to patients at risk of central nervous system disorders, or a related disorder. As used herein and unless otherwise indicated, the term “managing” encompasses preventing the recurrence of symptoms of central nervous system disorders in a patient who had suffered from a central nervous system disorder, lengthening the time the symptoms remain in remission in a patient who had suffered from central nervous system disorders, and/or preventing the occurrence of central nervous system disorders in patients at risk of suffering from central nervous system disorders.

In a specific embodiment, the central nervous system disorder to be prevented, treated and/or managed is Parkinson disease, Alzheimer disease, mild cognitive impairment, dementia, depression, defective long-term memory, Amyotrophic Lateral Sclerosis (ALS) or CNS trauma.

The invention encompasses methods of treating or preventing central nervous system disorders, preferably Parkinson disease or Alzheimer disease. In one embodiment, the methods of the invention are used to treat or prevent disorders related to movement, including, but not limited to, slow execution or bradykinesia, paucity of movement or akinesia, movement disorders that impair fine motor control and finger dexterity, and other manifestations of bradykinesia, such as, but not limited to, hypophonia and monotonic speech. In another embodiment, the methods of the invention are used to treat or prevent disorders related to muscular rigidity, including, but not limited to, a uniform increase in resistance to passive movement, interruptions to passive movement, and combinations of rigidity and dystonia. In a specific embodiment, methods of the invention are used to treat inflammation associated with Parkinson or related disease. In yet another embodiment of the invention, disorders resembling Parkinsonian tremor are treated or prevented by the methods of the invention, including but not limited to, tremors of the face, jaw, tongue, posture, and other tremors that are present at rest and that attenuate during movement. In another embodiment, the methods of the invention are used to treat or prevent disorders in gait, including, but not limited to, those resembling parkinsonian gait, shuffling, short steps, a tendency to turn en bloc, and festinating gait. In another embodiment of the invention, nonmotor symptoms are treated or prevented using the methods of the invention, including, but not limited to, disorders of mood, cognition, defective long-term memory, sensation, sleep, dementia, and depression. In other embodiment of the invention, secondary forms of parkinsonism are treated or prevented by the methods of the invention, including, but not limited to, drug induced parkinsonism, vascular parkinsonism, multiple system atrophy, progressive supranuclear palsy, disorders with primary tau pathology, cortical basal ganglia degeneration, parkinsonism with dementia, hyperkinetic disorders, chorea, Huntington disease, dystonia, Wilson disease, Tourette syndrome, essential tremor, myoclonus, and tardive movement disorders. In other embodiment of the invention, other central nervous system disorders are treated or prevented by the methods of the invention, including, but not limited to, Alzheimer disease, mild cognitive impairment, Amyotrophic Lateral Sclerosis (ALS) and CNS trauma.

Methods encompassed by this invention comprise administering one or more selective cytokine inhibitory drugs, or a pharmaceutically acceptable salt, solvate, hydrate, stereoisomer, clathrate, or prodrug thereof to a patient (e.g., a human) suffering, or likely to suffer, from central nervous system disorders.

Another method comprises administering 1) a selective cytokine inhibitory drug, or a pharmaceutically acceptable salt, solvate, hydrate, stereoisomer, clathrate, or prodrug thereof, and 2) a second active agent or active ingredient. Examples of selective cytokine inhibitory drugs are disclosed herein (see, e.g., section 4.1); and examples of the second active agents are also disclosed herein (see, e.g., section 4.2).

Administration of selective cytokine inhibitory drugs and the second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. A preferred route of administration for a selective cytokine inhibitory drug is orally. Preferred routes of administration for the second active agents or ingredients of the invention are known to those of ordinary skill in the art.

In one embodiment of the invention, the recommended daily dose range of a selective cytokine inhibitory drug for the conditions described herein lie within the range of from about 1 mg to about 10,000 mg per day, given as a single once-a-day dose, or preferably in divided doses throughout a day. More specifically, the daily dose is administered twice daily in equally divided doses. Specifically, a daily dose range should be from about 1 mg to about 5,000 mg per day, more specifically, between about 10 mg and about 2,500 mg per day, between about 100 mg and about 800 mg per day, between about 100 mg and about 1,200 mg per day, or between about 25 mg and about 2,500 mg per day. In managing the patient, the therapy should be initiated at a lower dose, perhaps about 1 mg to about 2,500 mg, and increased if necessary up to about 200 mg to about 5,000 mg per day as either a single dose or divided doses, depending on the patient's global response. In a particular embodiment, 3-(3,4-dimethoxy-phenyl)-3-(1-oxo-1,3-dihydro-isoindol-2-yl)-propionamide can be preferably administered in an amount of about 400, 800, 1,200, 2,500, 5,000 or 10,000 mg a day as two divided doses.

In another embodiment, the selective cytokine inhibitory drug is administered in conjunction with the second active agent. The second active agent is administered orally, intravenously or subcutaneously and once or twice daily in an amount of from about 1 to about 1,000 mg, from about 5 to about 500 mg, from about 10 to about 350 mg, or from about 50 to about 200 mg. The specific amount of the second active agent will depend on the specific agent used, the disorder being treated or managed, the severity and stage of the central nervous system disorder, and the amount(s) of selective cytokine inhibitory drugs and any optional additional active agents concurrently administered to the patient.

In certain embodiments, the prophylactic or therapeutic agents of the invention are cyclically administered to a patient. Cycling therapy involves the administration of a first agent for a period of time, followed by the administration of the agent and/or the second agent for a period of time and repeating this sequential administration. Cycling therapy can reduce the development of resistance to one or more of the therapies, avoid or reduce the side effects of one of the therapies, and/or improves the efficacy of the treatment.

In a preferred embodiment, prophylactic or therapeutic agents are administered in a cycle of about 24 weeks, about once or twice every day. One cycle can comprise the administration of a therapeutic or prophylactic agent and at least one (1) or three (3) weeks of rest. The number of cycles administered is from about 1 to about 12 cycles, more typically from about 2 to about 10 cycles, and more typically from about 2 to about 8 cycles.

4.4 Pharmaceutical Compositions And Single Unit Dosage Forms

Pharmaceutical compositions can be used in the preparation of individual, single unit dosage forms. Pharmaceutical compositions and dosage forms of the invention comprise a selective cytokine inhibitory drug, or a pharmaceutically acceptable salt, solvate, hydrate, stereoisomer, clathrate, or prodrug thereof. Pharmaceutical compositions and dosage forms of the invention can further comprise one or more excipients.

Pharmaceutical compositions and dosage forms of the invention can also comprise one or more additional active ingredients. Consequently, pharmaceutical compositions and dosage forms of the invention comprise the active ingredients disclosed herein (e.g., a selective cytokine inhibitory drug, or a pharmaceutically acceptable salt, solvate, hydrate, stereoisomer, clathrate, or prodrug thereof, and a second active ingredient). Examples of optional additional active ingredients are disclosed herein (see, e.g., section 4.2).

Single unit dosage forms of the invention are suitable for oral, mucosal (e.g., nasal, sublingual, vaginal, buccal, or rectal), or parenteral (e.g., subcutaneous, intravenous, bolus injection, intramuscular, or intraarterial), transdermal or transcutaneous administration to a patent. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18^{th} ed., Mack Publishing, Easton Pa. (1990).

Typical pharmaceutical compositions and dosage forms comprise one or more excipients. Suitable excipients are well known to those skilled in the art of pharmacy, and Non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise a selective cytokine inhibitory drug, or a pharmaceutically acceptable salt, solvate, hydrate, stereoisomer, clathrate, or prodrug thereof in an amount of from about 1 to about 1,200 mg. Typical dosage forms comprise a selective cytokine inhibitory drug, or a pharmaceutically acceptable salt, solvate, hydrate, stereoisomer, clathrate, or prodrug thereof in an amount of about 1, 2, 5, 10, 25, 50, 100, 200, 400, 800, 1,200, 2,500, 5,000 or 10,000 mg. In a particular embodiment, a preferred dosage form comprises 3-(3,4-dimethoxy-phenyl)-3-(1-oxo-1,3-dihydro-isoindol-2-yl)-propionamide in an amount of about 400, 800 or 1,200 mg. Typical dosage forms comprise the second active ingredient in an amount of 1 to about 1000 mg, from about 5 to about 500 mg, from about 10 to about 350 mg, or from about 50 to about 200 mg. Of course, the specific amount of the second active ingredient will depend on the specific agent used, the disorder being treated or managed, and the amount(s) of selective cytokine inhibitory drugs and any optional additional active agents concurrently administered to the patient.

4.4.1 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18^{th} ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, PA), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A preferred solid oral dosage form of the invention comprises a selective cytokine inhibitory drug, anhydrous lactose, microcrystalline cellulose, polyvinylpyrrolidone, stearic acid, colloidal anhydrous silica, and gelatin.

4.4.2 Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

4.4.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention. For example, cyclodextrin and its derivatives can be used to increase the solubility of a selective cytokine inhibitory drug and its derivatives. See, e.g., U.S. Pat. No. 5,134,127, which is incorporated herein by reference.

4.4.4 Topical and Mucosal Dosage Forms

Topical and mucosal dosage forms of the invention include, but are not limited to, sprays, aerosols, solutions, emulsions, suspensions, or other forms known to one of skill in the art. See, e.g., Remington's Pharmaceutical Sciences, 16^{th} and 18^{th} eds., Mack Publishing, Easton Pa. (1980 & 1990); and Introduction to Pharmaceutical Dosage Forms, 4^{th} ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed by this invention are well known given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16^{th} and 18^{th} eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

4.4.5 Kits

Typically, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a dosage form of a selective cytokine inhibitory drug, or a pharmaceutically acceptable salt, solvate, hydrate, stereoisomer, prodrug, or clathrate thereof. Kits encompassed by this invention can further comprise additional active ingredients. Examples of the additional active ingredients include, but are not limited to, those disclosed herein (see, e.g., section 4.2).

Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 5. EXAMPLES

The following studies are intended to further illustrate the invention without limiting its scope.

5.1 Pharmacology and Toxicology Studies

A series of non-clinical pharmacology and toxicology studies are performed to support the clinical evaluation of selective cytokine inhibitory drugs in human subjects. These studies are performed in accordance with internationally recognized guidelines for study design and in compliance with the requirements of Good Laboratory Practice (GLP), unless otherwise noted.

The pharmacological properties of 3-(3,4-dimethoxy-phenyl)-3-(1-oxo-1,3-dihydro-isoindol-2-yl)-propionamide, including activity comparisons with thalidomide, are characterized in in vitro studies. Studies examine the effects of 3-(3,4-dimethoxy-phenyl)-3-(1-oxo-1,3-dihydro-isoindol-2-yl)-propionamide on the production of various cytokines. In addition, a safety pharmacology study of 3-(3,4-dimethoxy-phenyl)-3-(1-oxo-1,3-dihydro-isoindol-2-yl)-propionamide is conducted in dogs and the effects of the compound on ECG parameters are examined further as part of three repeat-dose toxicity studies in primates.

5.2 Modulation of Cytokine Production

Inhibition of TNF-α production following LPS-stimulation of human PBMC and human whole blood by 3-(3,4-dimethoxy-phenyl)-3-(1-oxo-1,3-dihydro-isoindol-2-yl)-propionamide is investigated in vitro (Muller et al., Bioorg. Med. Chem. Lett. 9:1625-1630, 1999). The IC₅₀'s of 3-(3,4-dimethoxy-phenyl)-3-(1-oxo-1,3-dihydro-isoindol-2-yl)-propionamide for inhibiting production of TNF-α following LPS-stimulation of PBMC and human whole blood is measured.

In vitro studies suggest a pharmacological activity profile for 3-(3,4-dimethoxy-phenyl)-3-(1-oxo-1,3-dihydro-isoindol-2-yl)-propionamide that is similar to, but 5 to 50 times more potent than, thalidomide. The pharmacological effects of 3-(3,4-dimethoxy-phenyl)-3-(1-oxo-1,3-dihydro-isoindol-2-yl)-propionamide derive from its action as an inhibitor of cellular response to receptor-initiated trophic signals (e.g., IGF-1, VEGF, cyclooxygenase-2), and other activities. As a result, 3-(3,4-dimethoxy-phenyl)-3-(1-oxo-1,3-dihydro-isoindol-2-yl)-propionamide suppresses the generation of inflammatory cytokines, down-regulates adhesion molecules and apoptosis inhibitory proteins (e.g., cFLIP, clAP), promotes sensitivity to death-receptor initiated programmed cell death, and suppresses angiogenic response.

5.3 Toxicology Studies

The effects of 3-(3,4-dimethoxy-phenyl)-3-(1-oxo-1,3-dihydro-isoindol-2-yl)-propionamide on cardiovascular and respiratory function are investigated in anesthetized dogs. Two groups of Beagle dogs (2/sex/group) are used. One group receives three doses of vehicle only and the other receives three ascending doses of 3-(3,4-dimethoxy-phenyl)-3-(1-oxo-1,3-dihydro-isoindol-2-yl)-propionamide (400, 800, and 1,200 mg/kg/day). In all cases, doses of 3-(3,4-dimethoxy-phenyl)-3-(1-oxo-1,3-dihydro-isoindol-2-yl)-propionamide or vehicle are successively administered via infusion through the jugular vein separated by intervals of at least 30 minutes.

The cardiovascular and respiratory changes induced by 3-(3,4-dimethoxy-phenyl)-3-(1-oxo-1,3-dihydro-isoindol-2-yl)-propionamide are minimal at all doses when compared to the vehicle control group.

5.4 Studies in Parkinson Disease

The effects of selective cytokine inhibitory drugs in a model of Parkinson disease are investigated in mice. Male C57/BL6 mice are injected once daily for 7 days with MPTP (30 mg/kg, i.p.). Selective cytokine inhibitory drugs are administered once or twice daily for 14 days. On day 28, striata are removed, homogenized in perchloric acid, and centrifuged. The supernatant is removed and analyzed for dopamine and other monoamines such as serotonin by reverse-phase HPLC and electrochemical detection. Anti-Parkinson activity of selective cytokine inhibitory drugs is assessed in comparison to the reference compound, selegiline.

5.5 Studies in Alzheimer Disease

The effects of selective cytokine inhibitory drugs in a model of Alzheimer disease are investigated in rat PC12 pheochromocytoma cells. PC12 cells are cultured in the presence of dopamine, D1 dopamine receptor agonist, adenosine, adenosine A 2a receptor agonist, nicotine, or alpha 7 nicotinic acetylcholine receptor agonist and selective cytokine inhibitory drugs. After 24 hours, cellular supernatants are harvested and assayed for acetylcholinesterase activity by the Ellman method (Hawkins and Knittle, Anal Chem 44:416-417,1972). Suppression of acetylcholinesterase activity levels by selective cytokine inhibitory drugs is assessed in comparison to the reference compound tacrine.

5.6 Cycling Therapy in Central Nervous System Disorders

In a specific embodiment, selective cytokine inhibitory drugs are cyclically administered to patients with central nervous system disorders. Cycling therapy involves the administration of a first agent for a period of time, followed by the administration of the agent and/or the second agent for a period of time and repeating this sequential administration. Cycling therapy can reduce the development of resistance to one or more of the therapies, avoid or reduce the side effects of one of the therapies, and/or improves the efficacy of the treatment.

In a specific embodiment, prophylactic or therapeutic agents in an amount of about 400, 800 or 1200 mg are administered in a cycle of about 24 weeks, about once or twice every day. One cycle can comprise the administration of a therapeutic on prophylactic agent and at least one (1), two (2), or three (3) weeks of rest. The number of cycles administered is from about 1 to about 12 cycles, more typically from about 2 to about 10 cycles, and more typically from about 2 to about 8 cycles.

For example, on day 1 in a cycle of 24 weeks, blood product transfusion is administered to patients with Parkinson disease. On day 10, the administration of 800 mg/d of 3-(3,4-dimethoxy-phenyl)-3-(1-oxo-1,3-dihydro-isoindol-2-yl)-propionamide is started. On day 30, blood product transfusion is administered. On day 34, the administration of 800 mg/d of 3-(3,4-dimethoxy-phenyl)-3-(1-oxo-1,3-dihydro-isoindol-2-yl)-propionamide is stopped. On day 59, the administration of 400 mg/d of 3-(3,4-dimethoxy-phenyl)-3-(1-oxo-1,3-dihydro-isoindol-2-yl)-propionamide is begun.

Embodiments of the invention described herein are only a sampling of the scope of the invention. The full scope of the invention is better understood with reference to the attached claims.

What is claimed is:

1. A method of treating or preventing a central nervous system disorder, which comprises administering to a patient in need of such treatment or prevention a therapeutically or prophylactically effective amount of a selective cytokine inhibitory drug, or a pharmaceutically acceptable salt, solvate, or stereoisomer thereof.

2. A method of managing a central nervous system disorder, which comprises administering to a patient in need of such management a prophylactically effective amount of a selective cytokine inhibitory drug, or a pharmaceutically acceptable salt, solvate, or stereoisomer thereof.

3. The method of claim 1, wherein the central nervous system disorder is Parkinson disease; Alzheimer disease; mild cognitive impairment; Amyotrophic Lateral Sclerosis; CNS trauma; Alzheimer disease with parkinsonism; bradykinesia; alkinesia; movement disorder that impairs fine motor control and finger dexterity; hypophonia; monotonic speech; rigidity; dystonia; inflammation associated with Parkinson disease; tremor of the face, jaw, tongue or posture; parkinsonian gait; shuffling; short step; festinating gait; disorder of mood, cognition, sensation, or sleep; dementia; depression; defective long-term memory; drug induced parkinsonism; vascular parkinsonism; multiple system atrophy; progressive supranuclear palsy; disorder with primary tau pathology; cortical basal ganglia degeneration; parkinsonism with dementia; hyperkinetic disorder; chorea; Huntington disease; dystonia; Wilson disease; Tourette syndrome; essential tremor; myoclonus; or a tardive movement disorder.

4. The method of claim 2, wherein the central nervous system disorder is Parkinson disease; Alzheimer disease; mild cognitive impairment; Amyotrophic Lateral Sclerosis; CNS trauma Alzheimer disease with parkinsonism; bradykinesia; alkinesia; movement disorder that impairs fine motor control and finger dexterity; hypophonia; monotonic speech; rigidity; dystonia; inflammation associated with Parkinson disease; tremor of the face, jaw, tongue or posture; parkinsonian gait; shuffling; short step; festinating gait; disorder of mood, cognition, sensation, or sleep; dementia; depression; defective long-term memory; drug induced parkinsonism; vascular parkinsonism; multiple system atrophy; progressive supranuclear palsy; disorder with primary tau pathology; cortical basal ganglia degeneration; parkinsonism with dementia; hyperkinetic disorder; chorea; Huntington disease; dystonia; Wilson disease; Tourette syndrome; essential tremor; myoclonus; or a tardive movement disorder.

5. The method of claim 3, wherein the central nervous system disorder is Parkinson disease.

6. The method of claim 4, wherein the central nervous system disorder is Parkinson disease.

7. A method of treating or preventing a central nervous system disorder, which comprises administering to a patient in need of such treatment or prevention a therapeutically or prophylactically effective amount of a selective cytokine inhibitory drug, or a pharmaceutically acceptable salt, solvate, or stereoisomer thereof, and a therapeutically or prophylactically effective amount of at least one second active ingredient.

8. A method of managing a central nervous system disorder, which comprises administering to a patient in need of such management a prophylactically effective amount of a selective cytokine inhibitory drug, or a pharmaceutically acceptable salt, solvate, or stereoisomer thereof, and a therapeutically or prophylactically effective amount of at least one second active ingredient.

9. The method of claim 7, wherein the central nervous system disorder is Parkinson disease.

10. The method of claim 8, wherein the central nervous system disorder is Parkinson disease.

11. The method of claim 7, wherein the second active ingredient is a dopamine agonist, a monoamine oxidase inhibitor (MAO), a catechol-O-methyltransferase inhibitor (COMT), amantadine, an acetylcholinesterase inhibitor, an antiemetic, or an anti-inflammatory agent.

12. The method of claim 8, wherein the second active ingredient is a dopamine agonist, a monoamine oxidase inhibitor (MAO), a catechol-O-methyltransferase inhibitor (COMT), amantadine, an acetylcholinesterase inhibitor, an antiemetic, or an anti-inflammatory agent.

13. The method of any one of claims 1, 2, 7, or 8, wherein the stereoisomer of the selective cytokine inhibitory drug is an enantiomer.

14. The method of any one of claims 1, 2, 7, or 8, wherein the selective cytokine inhibitory drug is 3-(3,4-dimethoxy-phenyl)-3-(1-oxo-1,3-dihydro-isoindol-2-yl)propionamide.

15. The method of claim 14, wherein the selective cytokine inhibitory drug is the R or S enantiomer of 3-(3,4-dimethoxy-phenyl)-3-(1-oxo-1,3-dihydro-isoindol-2-yl)propionamide.

16. The method of any one of claims 1, 2, 7 or 8, wherein the selective cytokine inhibitory drug is cyclopropanecarboxylic acid {2-[1-(3-ethoxy-4-methoxy-phenyl)-2-methanesulfonyl-ethyl]-3-oxo-2,3-dihydro-1H-isoindol-4-yl}-amide.

17. The method of claim 16, wherein the selective cytokine inhibitory drug is the R or S enantiomer of cyclopropanecarboxylic acid {2-[1-(3-ethoxy-4-methoxy-phenyl)-2-methanesulfonyl-ethyl]-3-oxo-2,3-dihydro-1H-isoindol-4-yl}-amide.

18. The method of any one of claims 1, 2, 7 or 8, wherein the selective cytokine inhibitory drug has formula (I): 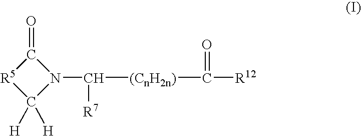
wherein n has a value of 1, 2, or 3;
R⁵ is o-phenylene, unsubstituted or substituted with 1 to 4 substitutents each selected independently from the group consisting of nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkyl of 1 to 10 carbon atoms, and halo;
R⁷ is (i) phenyl or phenyl substituted with one or more substitutents each selected independently of the other from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo, (ii) benzyl unsubstituted or substituted with 1 to 3 substitutents selected from the group consisting of nitro, cyano, trifluoromethyl, carbothoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo, (iii) naphthyl, and (iv) benzyloxy;
R¹² is —OH, alkoxy of 1 to 12 carbon atoms, or 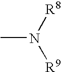
R⁸ is hydrogen or alkyl of 1 to 10 carbon atoms; and
R⁹ is hydrogen, alkyl of 1 to 10 carbon atoms, —COR¹⁰, or —SO₂R¹⁰, wherein R¹⁰ is hydrogen, alkyl of 1 to 10 carbon atoms, or phenyl.

19. The method of claim 18, wherein the selective cytokine inhibitory drug is an enantiomer of the compound having formula (I).

20. The method of any one of claims 1, 2, 7 or 8, wherein the selective cytokine inhibitory drug has formula (II): 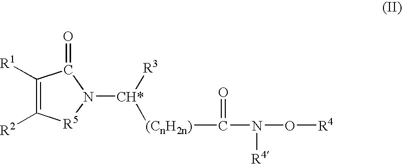
wherein each of R¹ and R², when taken independently of each other, is hydrogen, lower alkyl, or R¹ and R², when taken together with the depicted carbon atoms to which each is bound, is o-phenylene, o-naphthylene, or cyclohexene-1,2-diyl, unsubstituted or substituted with 1 to 4 substitutents each selected independently from the group consisting of nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkoxy of 1 to 10 carbon atoms, and halo;
R³ is phenyl substituted with from one to four substitutents selected from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, alkylthio of 1 to 10 carbon atoms, benzyloxy, cycloalkoxy of 3 to 6 carbon atoms, C₄-C₆-cycloalkylidenemethyl, C₃-C₁₀-alkylidenemethyl, indanyloxy, and halo;
R⁴ is hydrogen, alkyl of 1 to 6 carbon atoms, phenyl, or benzyl;
R^{4′} is hydrogen or alkyl of 1 to 6 carbon atoms;
R⁵ is —CH₂—, —CH₂—CO—, —SO₂—, —S—, or —NHCO—; and
n has a value of 0, 1, or 2.

21. The method of claim 20, wherein the selective cytokine inhibitory drug is an enantiomer of the compound having formula (II).

22. The method of any one of claims 1, 2, 7 or 8, wherein the selective cytokine inhibitory drug has formula (III): 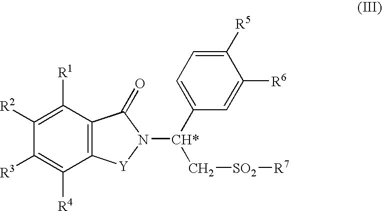
wherein the carbon atom designated * constitutes a center of chirality;
Y is C═O, CH2, SO₂, or CH₂C═O;
each of R¹, R², R³, and R⁴, independently of the others, is hydrogen, halo, alkyl of 1 to 4 carbon atoms, alkoxy of 1 to 4 carbon atoms, nitro, cyano, hydroxy, or —NR⁸R⁹; or any two of R¹, R², R³, and R⁴ on adjacent carbon atoms, together with the depicted phenylene ring are naphthylidene;
each of R⁵ and R⁶, independently of the other, is hydrogen, alkyl of 1 to 4 carbon atoms, alkoxy of 1 to 4 carbon atoms, cyano, or cycloalkoxy of up to 18 carbon atoms;
R⁷ is hydroxy, alkyl of 1 to 8 carbon atoms, phenyl, benzyl, or NR^{8′}R^{9′};
each of R⁸ and R⁹ taken independently of the other is hydrogen, alkyl of 1 to 8 carbon atoms, phenyl, or benzyl, or one of R⁸ and R⁹ is hydrogen and the other is —COR¹⁰ or —SO₂R¹⁰, or R⁸ and R⁹ taken together are tetramethylene, pentamethylene, hexamethylene, or —CH₂CH₂X¹CH₂CH₂— in which X¹ is —O—, —S— or —NH—; and
each of R^{8′} and R^{9′} taken independently of the other is hydrogen, alkyl of 1 to 8 carbon atoms, phenyl, or benzyl, or one of R^{8′} and R^{9′} is hydrogen and the other is —COR^{10′} or —SO₂R^{10′}, or R^{8′} and R^{9′} taken together are tetramethylene, pentamethylene, hexamethylene, or —CH₂CH₂X²CH₂CH₂— in which X² is —O—, —S—, or —NH—.

23. The method of claim 22, wherein the selective cytokine inhibitory drug is an enantiomer of said compound.

24. A method of reducing or avoiding an adverse effect associated with the administration of a second active ingredient in a patient suffering from a central nervous system disorder, which comprises administering to a patient in need of such reduction or avoidance an amount of the second active ingredient and a therapeutically or prophylactically effective amount of a selective cytokine inhibitory drug, or a pharmaceutically acceptable salt, solvate, or stereoisomer thereof.

25. A pharmaceutical composition comprising a selective cytokine inhibitory drug, or a pharmaceutically acceptable salt, solvate, or stereoisomer, thereof in an amount effective to treat, prevent or manage a central nervous system disorder, and a carrier.

26. A pharmaceutical composition comprising a selective cytokine inhibitory drug, or a pharmaceutically acceptable salt, solvate, or stereoisomer thereof, in an amount effective to treat, prevent or manage a central nervous system disorder, and a second active ingredient.

27. The pharmaceutical composition of claim 26, wherein the second active ingredient is a dopamine agonist, a monoamine oxidase inhibitor (MAO), a catechol-O-methyltransferase inhibitor (COMT), amantadine, an anticholinergic, an antiemetic, or an anti-inflammatory agent.
